(19) United States
(12) Patent Application Publication
(10) Pub. No.: US 2023/0333360 A1
(43) Pub. Date: Oct. 19, 2023

(54) Point-Of-Care Microscope for Real-Time Acquisition of Volumetric Histological Images In Vivo

(71) Applicant: The Trustees of Columbia University in the City of New York, New York, NY (US)

(72) Inventors: Elizabeth M.C. HILLMAN, New York, NY (US); Kripa B. PATEL, Long Island City, NY (US); Wenxuan LIANG, New York, NY (US)

(73) Assignee: The Trustees of Columbia University in the City of New York, New York, NY (US)

(21) Appl. No.: 18/207,445

(22) Filed: Jun. 8, 2023

Related U.S. Application Data
(63) Continuation of application No. PCT/US2021/063500, filed on Dec. 15, 2021.
(60) Provisional application No. 63/125,817, filed on Dec. 15, 2020.

(51) Int. Cl. G02B 21/00

(52) U.S. Cl. CPC G02B 21/0012

(57) ABSTRACT

A microscope routes excitation light through a first set of optical components so the excitation light is projected into a sample and forms a sheet of excitation light at an oblique angle. The position of the sheet varies depending on an orientation of the scanning element. The first set of optical components routes detection light back to the scanning element, which routes the detection light into a second set of optical components. The second set of optical components forms an intermediate image plane that is imaged onto a detector. In some embodiments, a folding mirror is disposed between the first and second sets of optical components. In some embodiments, an optically transparent spacer covers the first objective and is configured to press against tissue being imaged. This spacer sets the working distance for the first objective to capture a particular range of depths within the tissue.

### CROSS REFERENCE TO RELATED APPLICATIONS

This Application is a continuation of International Application PCT/US2021/063500, filed Dec. 15, 2021, which claims the benefit of U.S. Provisional Application 63/125,817, filed Dec. 15, 2020, each of which is incorporated herein by reference in its entirety.

### STATEMENT REGARDING FEDERALLY-SPONSORED RESEARCH

This invention was made with government support under grants NS108213, NS09429, NS104649, and CA236554 awarded by the National Institutes of Health, and under grants 1644869, 0801530, and 0954796 awarded by the National Science Foundation. The government has certain rights in the invention

### BACKGROUND

The microstructure and cellular organization of tissues has been used to differentiate tissue types and their normal and diseased states since the 19^{th} century. However, despite major medical advances, histological examination of tissues still requires tissues to be excised, processed, sliced, stained and then imaged. Biopsy sampling removes valuable tissue, may miss abnormalities and takes 20 minutes to days to process, prolonging procedures, driving up costs and preventing closed-loop decision-making.

Over 6 million biopsy procedures are performed in the US every year. However, the standard practice of excising, fixing and staining tissue for histopathological evaluation is costly and slow, delaying treatment while being confounded by sampling error. Although intraoperative frozen sections can provide results within 20 minutes, tissues suffer from freezing artifacts, poor quality sectioning, swollen cell morphologies and poor staining, with fatty tissues such as brain being particularly difficult to freeze and cut. The use of physical 2D histology slides also impedes evaluation workflow because pathologists must track features through multiple sections to gain a better understanding of 3D tissue morphology. Physical slides must be manually viewed through a microscope or else digitized prior to viewing, which requires additional time and resources.

Most importantly, however, both frozen and standard histology require physical removal of living tissue. For precious tissues such as the eye, heart or brain, conservative biopsy can lead to under-sampling and either misdiagnosis or incomplete surgical resection. The destructive nature of biopsies also means that they are almost never used for general surgical guidance, such as identification of tissue types or for screening large areas of the body. Ex vivo tissues also quickly lose features such as perfusion level and metabolic state which could provide valuable biomarkers of tissue health or disease state.

Confocal microendoscopy utilizes confocal scanning through a fibre-optic conduit to generate 2D images of in situ tissues, and can be achieved through the channel of an endoscope. However, current commercial embodiments of confocal microendoscopy rely on systemic injection of bright fluorescent dyes such as fluorescein to provide contrast, while their ability to only capture 2D images over a small field of view has proven challenging to interpret reliably. Although specificity could be improved with fluorescent markers that can selectively highlight disease, regulatory approval of such agents has proven prohibitively costly and complex in most cases. Two-photon fluorescence, second harmonic generation, fluorescence lifetime and stimulated Raman spectroscopy have also been demonstrated for both in vivo and bedside fresh tissue imaging and have revealed impressive intrinsic, or ‘label-free’, contrast. However, all of these approaches have limited acquisition speed making them intolerant of in-vivo motion and preventing real-time, large-area or 3D imaging, while their reliance on costly and/or high-power pulsed laser sources that have thus far restricted their use for in vivo clinical imaging, with a few exceptions.

### SUMMARY OF THE INVENTION

One aspect of the invention is directed to a first imaging apparatus that includes first and second sets of optical components, a scanning element, a folding mirror, a light detector array, and a third objective. The first set of optical components has a proximal end and a distal end, and includes a first objective disposed at the distal end of the first set of optical components. The first objective has a magnification between 10× and 70× and a numerical aperture between 0.5 and 1.1. The second set of optical components has a proximal end and a distal end, and includes a second objective disposed at the proximal end of the second set of optical components. The scanning element is disposed proximally with respect to the proximal end of the first set of optical components and distally with respect to the distal end of the second set of optical components. The scanning element is arranged to route excitation light through the first set of optical components in a proximal to distal direction so that the excitation light is projected into a sample that is positioned distally beyond the distal end of the first set of optical components. The excitation light that is projected into the sample forms a sheet of excitation light at an oblique angle, and a position of the sheet varies depending on an orientation of the scanning element. The first set of optical components routes detection light from the sample in a distal to proximal direction back to the scanning element. The scanning element routes the detection light so that the detection light will pass through the second set of optical components in a distal to proximal direction, so that the second set of optical components forms an intermediate image plane at a position that is proximally beyond the proximal end of the second set of optical components. The folding mirror is disposed proximally with respect to the proximal end of the first set of optical components and distally with respect to the distal end of the second set of optical components. The third objective is arranged to route light arriving from the intermediate image plane towards the light detector array.

In some embodiments of the first imaging apparatus, the folding mirror is positioned between the scanning element and the distal end of the second set of optical components.

In some embodiments of the first imaging apparatus, the first objective has a magnification between 50× and 70×, a numerical aperture between 0.9 and 1.1, and an effective focal length between 2.5 and 3.5 mm, and the second objective has a magnification between 40× and 60×, a numerical aperture between 0.65 and 0.85, and an effective focal length between 3 and 5 mm.

Optionally, in the embodiments described in the previous paragraph, the first objective has a magnification of 60×, a numerical aperture of 1.0, and an effective focal length of 3 mm. Optionally, in the embodiments described in the previous paragraph, the second objective has a magnification of 50 ×, a numerical aperture of 0.75, and an effective focal length of 4 mm. Optionally, in the embodiments described in the previous paragraph, the first set of optical components includes at least one Plössl lens. Optionally, in the embodiments described in the previous paragraph, the first set of optical components comprises a 12.7 mm diameter 38.1 mm EFL achromat and a Plössl lens comprising two 12.7 mm diameter 50.8-mm-EFL achromats. Optionally, in the embodiments described in the previous paragraph, the second set of optical components includes at least one Plössl lens. Optionally, in the embodiments described in the previous paragraph, the second set of optical components comprises a Plössl lens made of two 1″ diameter 101.6-mm-EFL achromats and a 1″ diameter 76.2-mm-EFL achromat. Optionally, in the embodiments described in the previous paragraph, the first set of optical components comprises a telescope with a 1.5× magnification, and the second set of optical components comprises a telescope with a 1.5× magnification. Optionally, in the embodiments described in the previous paragraph, the third objective has a magnification between 15× and 25× and a numerical aperture between 0.65 and 0.85. Optionally, in the embodiments described in the previous paragraph, the third objective has a magnification of 20× and a numerical aperture of 0.75.

Another aspect of the invention is directed to a second imaging apparatus that includes a first and second sets of optical components, a scanning element, a light detector array, a third objective, and an optically transparent spacer. The first set of optical components has a proximal end and a distal end, and includes a first objective disposed at the distal end of the first set of optical components. The second set of optical components has a proximal end and a distal end, and includes a second objective disposed at the proximal end of the second set of optical components. The scanning element is disposed proximally with respect to the proximal end of the first set of optical components and distally with respect to the distal end of the second set of optical components. The scanning element is arranged to route excitation light through the first set of optical components in a proximal to distal direction so that the excitation light is projected into a sample that is positioned distally beyond the distal end of the first set of optical components. The excitation light that is projected into the sample forms a sheet of excitation light at an oblique angle, and a position of the sheet varies depending on an orientation of the scanning element. The first set of optical components routes detection light from the sample in a distal to proximal direction back to the scanning element. The scanning element is further arranged to route the detection light so that the detection light will pass through the second set of optical components in a distal to proximal direction, so that the second set of optical components forms an intermediate image plane at a position that is proximally beyond the proximal end of the second set of optical components. The third objective arranged to route light arriving from the intermediate image plane towards the light detector array. The optically transparent spacer is positioned and configured to cover the first objective and to press against tissue being imaged.

In some embodiments of the second imaging apparatus, the optically transparent spacer sets a working distance for the first objective to capture a 50-350 μm depth range into the tissue.

In some embodiments of the second imaging apparatus, the optically transparent spacer is incorporated into a cap that provides a watertight seal between the optically transparent spacer and a distal end of the first objective. Optionally, these embodiments may further comprise a quantity of a medium positioned between the optically transparent spacer and the first objective, wherein the medium has a refractive index selected to match an immersion medium of the first objective, and wherein the quantity of medium optically couples the optically transparent spacer to the first objective, and wherein the cap provides a water-tight seal.

In some embodiments of the second imaging apparatus, the optically transparent spacer is formed from a solid medium with a refractive index matching a required immersion medium of the first objective. In some embodiments of the second apparatus, the optically transparent spacer has an external surface positioned between 25 and 250 proximal to a primary focal plane of the first objective. In some embodiments of the second apparatus, the optically transparent spacer permits fast 3D imaging of a sample that is gradually moved across an external surface of the spacer permitting stitching of a contiguous 3D image of the sample. In some embodiments of the second apparatus, the first objective has a magnification between 10× and 70× and a numerical aperture between 0.5 and 1.1.

### BRIEF DESCRIPTION OF THE DRAWINGS

FIG. 1 depicts one embodiment of a swept confocally aligned planar excitation microscope.

FIG. 2 depicts another embodiment of a swept confocally aligned planar excitation microscope.

FIG. 3A shows the geometry of the single objective light sheet excitation and emission in intact tissues for the FIG. 1 and FIG. 2 embodiments.

FIG. 3B shows how single or dual color yz slices are collected along the scan direction (x) to create an oblique volume.

FIGS. 4A and 4B show the theoretical operational range of stigmatic imaging for the FIG. 1 and FIG. 2 embodiments, respectively.

FIG. 5 shows the Optical resolution of the FIG. 2 embodiment at different focal depths.

FIG. 6 depicts an example of an imaging cap designed to cover the distal end of the first objective in both the FIG. 1 and FIG. 2 embodiments.

Various embodiments are described in detail below with reference to the accompanying drawings, wherein like reference numerals represent like elements.

### DESCRIPTION OF THE PREFERRED EMBODIMENTS

Here, we demonstrate a small form-factor swept confocally aligned planar excitation microscope, termed MediSCAPE, that enables in situ, volumetric histological imaging of living tissue, in real-time and without the need for tissue excision. The high-speed 3D imaging performance of MediSCAPE withstands in vivo motion and enables roving 3D image acquisition, which combined with 3D stitching permits the contiguous analysis of large tissue areas. MediSCAPE's high sensitivity, even for weak intrinsic fluorescence, permits real-time multispectral 3D imaging of clinically relevant tissue architectures in intact, in situ living tissues without the need for exogenous staining. MediSCAPE is demonstrated in diverse in vivo and fresh mouse and human tissues, confirming the robust visualization of histoarchitectural structures, disease markers and in-vivo perfusion and tissue function.

MediSCAPE is an in vivo imaging methodology based on swept confocally aligned planar excitation (SCAPE) microscopy which permits rapid, non-destructive, in situ examination of tissues on a microscopic level without the need for excision, processing and staining. The approach has the potential to provide real-time, intraoperative feedback enabling closed-loop treatment decisions including assessment of surgical margins and surveillance of large tissue areas to guide biopsy site selection. MediSCAPE's non-destructive nature may also make it valuable for a range of non-pathological applications such as ‘tissue typing’ or perfusion assessment during robotic or orthopedic surgeries. Rapid, in situ histopathology could also be transformative for the evaluation of organs donated for human transplant, particularly kidneys which are the most commonly transplanted and suffer from high inter-observer variability.

While the past decade has seen the emergence of a range of new ‘bedside’ fresh tissue histopathology approaches that reduce the need for tissue sectioning, these methods image samples ex vivo, and often require additional tissue processing. For example, recent innovations applying light-sheet imaging to ex vivo tissues are proving the value of 3D acquisition and visualization. However, their use in vivo is limited by tissue staining and clearing steps, as well as the need to physically move tissue to form a 3D image. Recent demonstrations of two-photon and stimulated Raman spectroscopy for bedside imaging of unprocessed fresh tissue samples have revealed impressive intrinsic contrast. Still, their limited speed and reliance on costly high-power pulsed laser sources have thus far restricted their use for in vivo clinical imaging, with a few exceptions. Moreover, since imaging excised tissues removes constraints on utilizing a wide range of selective dyes and labels, simpler techniques such as MUSE microscopy are a viable alternative.

SCAPE microscopy is a high-speed 3D, single-objective light sheet approach that we originally developed for imaging cellular-level function and structure in model organisms. However, it delivers two unique capabilities which make it ideal for imaging human tissue in a clinical setting. 1) SCAPE can acquire 3D images of intact tissue at over 10 volumes per second, permitting near-instantaneous capture of 3D multi-layered structures in in situ tissues equivalent to a full box of histology slides. This high speed also provides tolerance of natural movements unavoidable in human surgical settings, and permits ‘roving’ acquisitions in which a continuum of volumetric images can be acquired and stitched together into continuous 3D histopathology spanning large areas of intact tissues. 2) Despite its high speed, SCAPE also has high sensitivity, permitting detection of intrinsic fluorophores already present in most tissues, removing the need for exogenous dyes, while also not requiring the use of high-power pulsed lasers, greatly facilitating clinical translation and in situ human use.

MediSCAPE imaging was demonstrated in a range of in vivo and freshly excised mouse and human tissues, comparing structures visualized to gold standard histology on the same tissues. We highlight that MediSCAPE's volumetric data allows examination of tissue microarchitecture in its natural 3D context with the ability to scroll through cross-sections at any arbitrary angle, greatly improving interpretability. We showed that video-rate volumetric imaging speeds permit 3D stitching of ‘roving scans’ over large tissue areas and overcome motion artifacts by imaging the in vivo beating mouse heart. We also demonstrated the use of exogenous dyes, including proflavine and fluorescein sodium, to highlight familiar cellular-components visible in H&E histology.

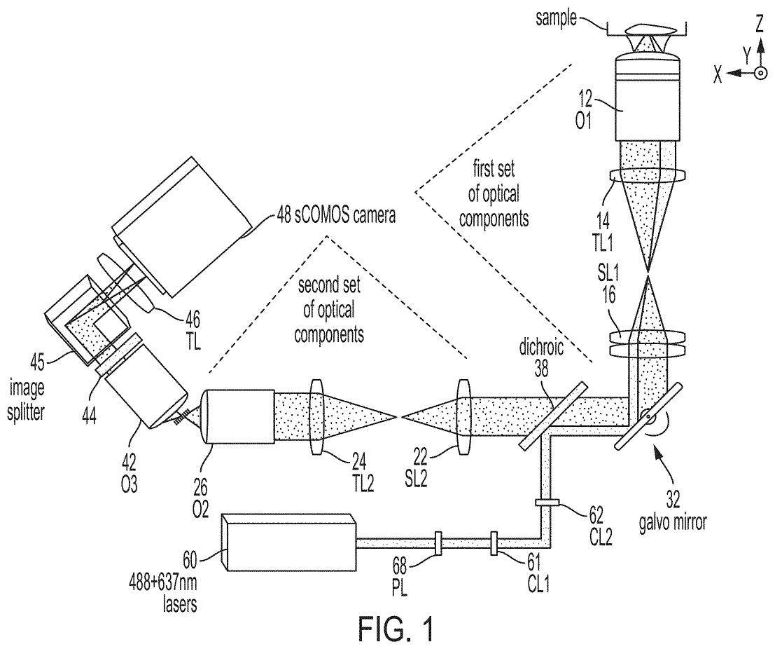

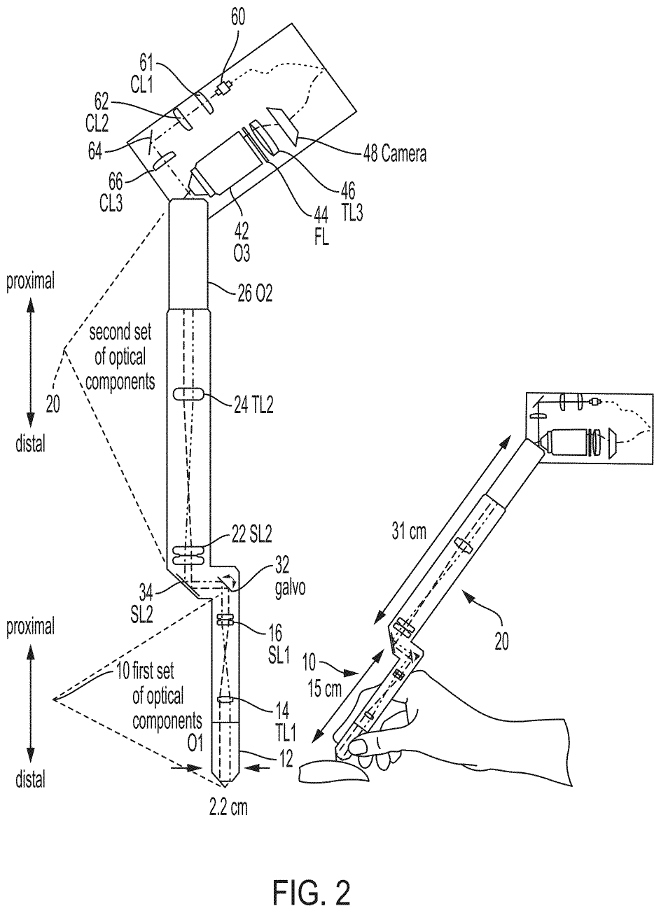

Datasets were acquired using two MediSCAPE embodiments: an optimized benchtop system (FIG. 1), and a novel miniaturized version of MediSCAPE with a form factor amenable to intraoperative human use with only modest compromises in performance (FIG. 2). This miniaturized design demonstrates MediSCAPE's potential to be used for in vivo tissue imaging in accessible orifices, as well as during laparoscopic, robotic and open-field surgeries. All images shown were acquired using affordable, visible, continuous wave laser light sources (488 nm and 637 nm), with equivalent illumination levels to FDA-approved confocal endomicroscopy. These demonstrations suggest that MediSCAPE could provide a new paradigm for microscopy-based intrasurgical guidance.

FIG. 1 depicts the benchtop embodiment, suitable for imaging in vivo rodent models and fresh ex vivo mouse and human tissue samples, such as resected kidney. This embodiment is similar to the configuration disclosed in U.S. Pat. No. 10,061,111 (which is incorporated herein by reference in its entirety), but with the addition of both 488 and 637 nm OBIS lasers for excitation, and 3-axis motorized stages (Thorlabs DDSM50 and MTS25-Z8) for stage-scanning when needed. Dual color imaging is achieved using a home-built image splitter in front of the camera to collect spectrally-resolved emission images in parallel. All imaging with this system was performed in an inverted configuration, with water between the objective and the coverslip upon which the sample was placed.

FIG. 2 depicts the miniaturized MediSCAPE design. This compact ‘unfolded’ design with a narrow and elongated imaging head, produces a form factor that could be boom-mounted and hand-guided for clinical use, with the only trade-off being a modestly reduced field of view. Images on this miniaturized FIG. 2 system were captured in an upright configuration, with a coverslip flattening the tissue when needed. Imaging parameters for all data shown is listed in Table 2.

In both the FIG. 1 and FIG. 2 embodiments, MediSCAPE uses an oblique light sheet to illuminate the sample and collects emitted fluorescence back through the same, single, stationary high numerical aperture (NA) objective lens 12. A galvanometer mirror 32 within the system both sweeps the light-sheet from side to side (along x), and descans the returning fluorescence, mapping it onto a stationary conjugate oblique image plane which is then focused onto a camera 48 (e.g., an sCMOS camera such as the Andor Zyla 4.2+). Planes corresponding to oblique yz′ sections are acquired by the camera 48 as the galvanometer mirror 32 sweeps the sheet in x to generate a volumetric image. Since all components of the system remain stationary except the galvo mirror, which sweeps at one line per volume, imaging speed is limited only by camera read-out rate. The volume acquisition rate is thus determined by the number of x-steps spanning each volume as well as the number of camera rows acquired on the camera (which corresponds to the depth in z′ imaged), with fewer rows permitting faster read-out e.g. ˜2,000 frames per second for 100 rows on a standard sCMOS camera. In all systems, dual colour imaging was achieved using a home-built image splitter in front of the camera, splitting images across columns to collect spectrally-resolved emission images in parallel without a reduction in speed.

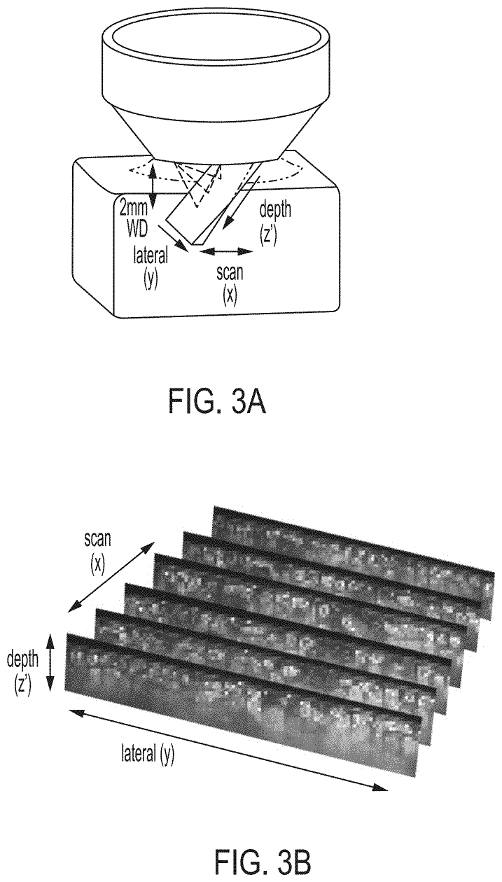

Images were acquired in one of three imaging paradigms: 1) galvanometer mirror-based scanning of the light sheet for volumetric imaging of a stationary sample (over a ˜1×1 mm² xy field of view), 2) roving scanning during which the sample was manually moved during continuous mirror-based volumetric imaging and 3) stage-scanning of the sample along x with a static light sheet (galvanometer stationary). Larger fields of view were generated by stitching either volumes from roving scans of in vivo tissue, or sequential stage scans of ex vivo tissue. Stage scanning is well suited for rapid 3D scanning just below the surface (e.g., at depths of 50-350 μm) of large tissue sections or slabs. When stage scanning is implemented, the scanning element 32 is held in a fixed position, and the sample is translated with respect to the entire microscope (or vice versa). Stage scanning may be implemented with the probe in contact or placed against glass or another flat material and imaged from above or below. Ex-vivo tissues could more readily be stained with a range of different dyes and stains as well as being imaged via autofluorescence. FIG. 3A shows the geometry of SCAPE's single objective light sheet excitation and emission in intact tissues. An oblique light sheet illuminates a single plane along yz′ while fluorescence emission is collected through the same sample objective.

FIG. 3B shows how single or dual color yz′ slices are collected along the scan direction (x) to create an oblique volume.

In the FIG. 1 embodiment, 488 and 637nm laser light is passed through a 30° Powell lens 68 (PL) and 50 mm and 75 mm cylindrical lenses 61, 62 (CL) to form a uniform light sheet. This sheet is brought into the primary optical path with a dichroic 38 and positioned off-center in x to form an oblique light sheet at the sample objective 12 (O1), here a water 20×1.0 NA Olympus objective with 2 mm working distance. The resulting plane of fluorescence is collected through the same objective 12 and mapped onto a stationary intermediate image plane between the secondary objective 26 (O2) (Nikon 20×, 0.75 NA, air) and tertiary objective 42 (Nikon 10×, 0.45 NA, air) by two telescopes arranged in a 4f-configuration. Telescope 1 is composed of a 75 mm Plössl lens 16 (SL1) and 150 mm achromat 14 (TL1) for 2× magnification, and telescope 2 is composed of a 60 mm achromat 22 (SL2) and a 100 mm achromat 24 (TL2) for 1.67× magnification. The stationary image plane is then imaged onto an sCMOS camera 48, with a homebuilt image splitter 45 providing spectral separation into two emission channels when needed. A 70 mm focal length tube lens 46 (TL) giving a final magnification of 4.6× was used for all bench-top datasets, except for data from Movie 2 (described below), which used a 70-200mm variable focal length TL. During mirror-based scanning, a volume is imaged by using the galvanometer mirror to sweep the light sheet and simultaneously descan the emitted fluorescence onto the stationary image plane, as described in U.S. Pat. No. 10,061,111.

The majority of previous SCAPE microscopy systems have been used as benchtop instruments for scientific research, and thus have not required significant miniaturization. However, in order for MediSCAPE to be translated into clinical use, some miniaturization and simplification of the design with respect to the FIG. 1 embodiment can be advantageous.

The FIG. 2 embodiment described herein is a MediSCAPE design which maintains imaging performance, while having a more compact form factor that makes it suitable for intraoperative use in open surgical fields as well as for oral and gynecological examination. With a smaller, custom primary objective lens, this same design could be used in smaller orifices such as the ear, nose and throat, and for arthroscopy and laparoscopy (particularly in combination with robotic surgery).

In the FIG. 2 embodiment, images are acquired through a narrow 15 cm long conduit that is 2.2 cm in diameter (with the diameter limited only by our use of a commercial 60× objective lens). After a small bend to accommodate the system's galvanometer mirror, the conduit then continues in-line for 31 cm with a <3 cm diameter, attaching to a scan head which holds additional optics and the system's camera which connects to a separate computer. The system's camera and laser sources could be located at a distance from the imaging head, relayed via fiber optic coupling if needed.

The FIG. 2 embodiment is portable and amenable to hand-held in vivo imaging, while maintaining cellular-level resolution and a practical depth range and lateral field of view. Optionally, it may be constructed using the components listed in Table 1. This whole unit could be mounted on a surgical microscope frame permitting flexible hand-guided movement of the imaging head within the surgical field, with small scale movements and scanning stabilized electromechanically. Rod and grin lenses could extend the narrower part of the imaging arm to be more compatible with laparoscopic insertion, while forward or side-facing MediSCAPE imaging could be added for intraluminal imaging.

Some embodiments use a water immersion primary objective, with a 2 mm working distance (WD). For clinical use, a sterile sheath may be fabricated to cover the imaging head, which would incorporate an optically transparent spacer to provide stabilization of the tissue being imaged, while also ensuring the optimal working distance for the objective to capture a 200-300 μm depth range into the tissue. In some embodiments the depth range is 50-350 μm.

Further miniaturization of MediSCAPE is possible, for example by combining fiber optic bundle-based detection with a distal-end scan head using MEMS mirrors and GRIN lenses. However, this implementation would likely sacrifice image quality and field of view, and would primarily serve gastroenterological endoscopy applications.

For in vivo clinical use, the FIG. 2 embodiment provides a narrower, longer imaging head that permits manoeuvring in the surgical field without obscuring the surgeon's access to the field. As shown in FIG. 2, this feature was achieved using unfolding mirror 34 to unfold the FIG. 1 system's usually orthogonal telescopes, and positioning the galvanometer mirror 32 within the primary elongated beam path. A smaller diameter 60× 1.0 NA water immersion primary objective lens 12 (O1 ) was chosen, while 2″ diameter lenses in the FIG. 1 system were replaced with 12 mm diameter optics. To provide more mechanical stability to alignment, the laser illumination is introduced via a single mode fibre and then directed into objective 26 (O2), simplifying the imaging head while enabling the image rotation objectives and laser launch to all be rigidly mounted on a plate distal to the imaging head.

The FIG. 2 embodiment illuminates the tissue with an oblique light sheet, incident from a primary objective lens 12 (O1). Fluorescence excited by this sheet is collected through the same objective lens. A lens telescope 14, 16 (TL1 and SL1) maps light between O1 12 and a galvanometer mirror 32 which is used to sweep the excitation sheet from side to side in the sample, and to redirect the returning light into a second imaging telescope 24, 22 (TL2 and SL2) to a secondary objective lens 26 (O2). This lens creates an intermediate image of the sample, which remains stationary with respect to the scanning light sheet thanks to the descanning function of the galvanometer mirror 32. The oblique image of the light sheet in this intermediate image space is relayed onto a camera 48 by a third, obliquely aligned objective lens 42 (O3). In the embodiment depicted in FIG. 2, this intermediate image space is also used as a place to introduce the excitation light.

The primary objective 12 (O1) should provide as high NA and long working distance (WD) as possible. We replaced the 20× 1.0 NA Olympus XLUMPLFLN2OW used in the FIG. 1 system, which has a diameter of 30 mm, for the much smaller 60× Olympus water-immersion objective (LUMPLFLN 60XW), which is 22 mm in diameter and has a 3 mm effective focal length (EFL), 1.0 full NA and 2 mm WD in water. The primary impact of moving to this higher magnification objective is a reduction in the system's usable field of view (FOV) from 1.0 mm to 0.4 mm. In alternative embodiments, the primary objective has a magnification between 50× and 70×, a numerical aperture between 0.9 and 1.1, and an effective focal length between 2.5 and 3.5 mm.

To isotropically replicate the 3D sample volume imaged by O1 to the intermediate 3D image formed by O2, the semi-aperture acceptance angle of O2 should be no less than that of O1. Therefore, we chose as O2 a 50× Mitutoyo plano apochromatic objective (#58-237, Edmund) with 0.75 NA (in air) and a 4 mm EFL. This objective features a 5.2 mm WD, allowing sufficient space and flexibility both for re-imaging the stationary intermediate image through O3 and for launching of the excitation sheet. In alternative embodiments, O2 has a magnification between 40× and 60×, a numerical aperture between 0.65 and 0.85, and an effective focal length between 3 and 5 mm.

The FIG. 2 embodiment has a first set of optical components 10 having a proximal end and a distal end. The first set of optical components 10 includes a first objective 12 disposed at the distal end of the first set of optical components. The first objective 12 has a magnification between 10× and 70× and a numerical aperture between 0.5 and 1.1. This embodiment also has a second set of optical components 20 having a proximal end and a distal end. The second set of optical components 20 includes a second objective 26 disposed at the proximal end of the second set of optical components 20. In some (but not all) versions of the FIG. 2 embodiment, the first objective 12 has a magnification between 50× and 70×, a numerical aperture between 0.9 and 1.1, and an effective focal length between 2.5 and 3.5 mm; and the second objective 26 has a magnification between 40× and 60×, a numerical aperture between 0.65 and 0.85, and an effective focal length between 3 and 5 mm.

A scanning element 32 is disposed proximally with respect to the proximal end of the first set of optical components 10 and distally with respect to the distal end of the second set of optical components 20. The scanning element 32 is arranged to route excitation light through the first set of optical components 10 in a proximal to distal direction so that the excitation light is projected into a sample that is positioned distally beyond the distal end of the first set of optical components 10. The excitation light that is projected into the sample forms a sheet of excitation light at an oblique angle, and the position of the sheet varies depending on the orientation of the scanning element 32. The first set of optical components 10 routes detection light from the sample in a distal to proximal direction back to the scanning element 32. The scanning element routes the detection light so that the detection light will pass through the second set of optical components 20 in a distal to proximal direction, so that the second set of optical components 20 forms an intermediate image plane at a position that is proximally beyond the proximal end of the second set of optical components 20.

A folding mirror 34 is disposed proximally with respect to the proximal end of the first set of optical components 10 and distally with respect to the distal end of the second set of optical components 20. In the embodiment illustrated in FIG. 2, the folding mirror 34 is positioned between the scanning element 32 and the distal end of the second set of optical components 20. But in alternative embodiments (not shown), the positions of the scanning element 32 and the folding mirror 34 are swapped, in which case the folding mirror 34 would be positioned between the scanning element 32 and the proximal end of the first set of optical components 10.

A third objective 42 is arranged to route light arriving from the intermediate image plane towards a light detector array 48.

Experimental and theoretical characterization reveals equivalent or even superior resolution and light efficiency of the FIG. 2 system compared to FIG. 1 system, with resolution close to the beam waist of 0.811±0.123 μm (y), 1.07±0.115 μm (x), and 2.10±0.479 μm (z), with the only trade-off being a modestly reduced field of view (˜1 mm×1 mm x-y for systems A and B v/s ˜0.4×0.6 x-y for the FIG. 2 system).

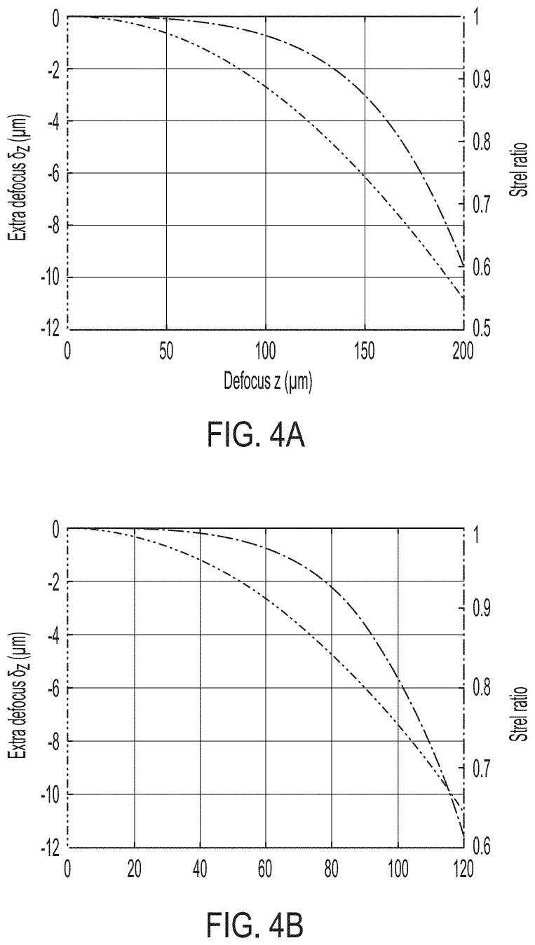

FIGS. 4A and 4B show the theoretical operational range of stigmatic imaging afforded by this O1-O2 combination of the FIG. 1 design and the FIG. 2, respectively. The Strehl ratio and coefficient of defocus of an on-axis point source at varying defocusing distances were calculated for both embodiments. As shown, the O1-O2 combination can accommodate a defocusing range of about ±80 μm (with Strehl ratio 0.9 and extra defocus less than 5 μm) which provides a ˜160 μm axial range, sufficient for optical imaging of biological tissue. The O1-O2 combination chosen for the FIG. 2 embodiment provides a good trade-off between operational range and compactness.

In the FIG. 2 embodiment, the scan and tube lenses 16, 14 (SL1 and TL1) for a relay telescope between O1 to the galvo are chosen to meet the following criteria: 1) the outer diameter should be as compact as possible; 2) the entire 4f-system should form a long enough handheld portion for easy maneuverability; 3) the focal length of TL1 should be long enough to reach the back focal plane of O1 12, which is located 19.1 mm inside the objective, but not so long that there is cropping of marginal rays from the edges of O1's FOV (around 400 μm in diameter); 4) the 6-mm-diameter back pupil of O1should be de-magnified onto the galvanometer mirror without aperture loss. Factoring in these considerations, we chose to use a 12.7 mm diameter 38.1 mm EFL achromat as TL1 and a Plössl lens comprising two 12.7 mm diameter 50.8-mm-EFL achromats as SL1. The second relay telescope (SL2 and TL2) is situated further away from the sample space, so we relaxed their physical diameter constraint to 1 inch, choosing a Plössl lens made of two 1″ diameter 101.6-mm-EFL achromats as SL2 and a 1″ diameter 76.2-mm-EFL achromat as TL2. For all Plössl assemblies, separations between the constituent achromats were first modeled in OpticStudio 16.5 (Zemax LLC) and Solidworks 2016 (Dassault Systemes), and then practically controlled with sub-mm precision by stacking optical spacers 0.4 mm or 1.0 mm in thickness (SM1S01, SM05S1M, and SM1S1M, Thorlabs).

Both of these telescopes have 1.5× magnification, but the ratio of the EFLs for O1 12 (3 mm) and O2 26 (4 mm) yields an effective magnification from the sample to the intermediate image of 1.33, meeting the ‘perfect 3D imaging condition’ for re-imaging based on our use of water-immersion (n=1.33) and air (n=1) objectives for O1 and O2 respectively. The second telescope is folded close to the galvo mirror using a 90-degree silver mirror 34 to form a linear configuration as shown in FIG. 2.

To help miniaturize this FIG. 2 design, the location at which excitation light is launched into the system was moved from between SL2 22 and the galvo 32 (as in the benchtop design shown in FIG. 1) to instead enter at O2 26. This approach effectively creates the light sheet at the intermediate image, and relays it to the sample in the same way as the returning image is relayed from the sample to the intermediate image as described in US 2019/0317312, which is incorporated herein by reference in its entirety.

To form the sheet, laser light (488 nm) from a single-mode fiber 60 (SM450, Thorlabs) with 2.8 μm mode-field diameter was collimated by a 15 mm EFL aspheric lens into a Gaussian beam —3.33 mm in 1/e2 waist. This Gaussian beam was expanded by —3.3x by a cylindrical 4f-system 61, 62 (CL1 and CL2), and then focused by a 50-mm-EFL cylindrical lens 66 (CL3), all along the x-direction, to generate an elliptical Gaussian beam. The beam waist of the sheet was carefully aligned to coincide with the focal plane of O2 26 and launched with a ˜39° oblique angle into O2, corresponding to a —2.5 mm lateral beam offset on the back aperture of O2 26.

A Nikon Plan Apo λ 20× 0.75 NA objective was chosen as the detection objective O3 42. It was paired with a tube lens 46 (TL3) of appropriate focal length (e.g., 35 mm EFL for tissue imaging, or 135 mm EFL for resolution calibration) to magnify the intermediate image onto an sCMOS camera 48 (Andor Zyla 4.2+). Since O3 is corrected for a 170 μm thick coverslip, a coverslip mount was fabricated using 3D printing and installed in front of O3 to minimize spherical aberration. In alternative embodiments, O3 has a magnification between 15× and 25× and a numerical aperture between 0.65 and 0.85.

Resolution was characterized by imaging 200 nm diameter fluorescent beads embedded in 1% agarose gel. A 135 mm EFL tube lens (SAL135F18Z, Sony) was used as TL3 to provide an overall ˜18× magnification from the sample to the camera (Zyla 4.2+, 6.5 μm pixel size). Sampling densities were confirmed by manually translating the sample 100 μm along x-, y- or z-directions and quantifying bead displacements, yielding Δₓ=0.337 μm, Δ_{y}=0.371 μm, and Δ_{z}=0.286 μm respectively. The sheet angle at the sample was calibrated to be 39.5°. After deskewing the MediSCAPE data, the FWHM resolution of the system was estimated to be 0.811±0.123 μm (y), 1.07±0.115 μm (x), and 2.10±0.479 μm (z) near the sheet waist. While the x and y resolution do not change substantially with depth, the z-resolution decreases with distance from the beam waist, as expected.

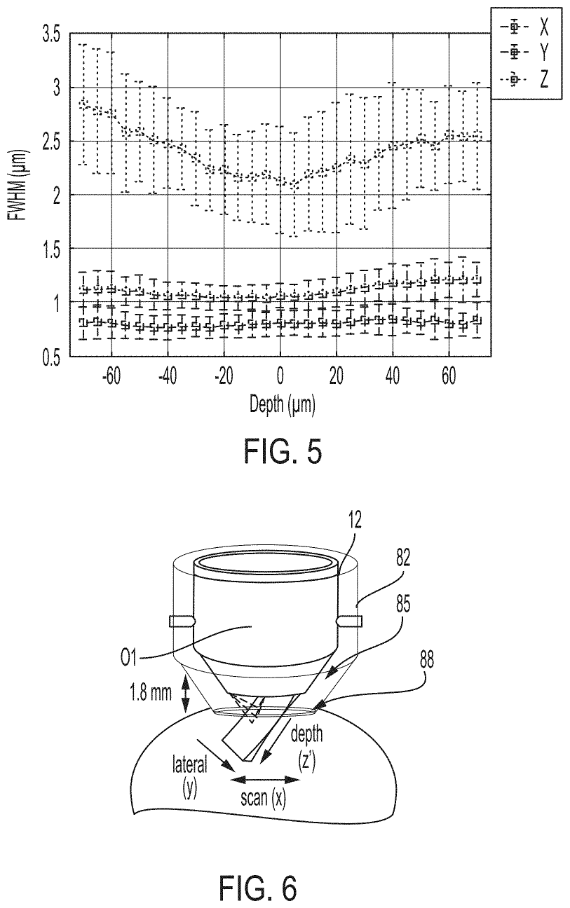

FIG. 5 shows the Optical resolution of the FIG. 2 embodiment of MediSCAPE at different focal depths. Around 6,300 beads were extracted from the skew-corrected three-dimensional data, and their FWHM size along all three directions were estimated. The beads were then grouped according to their depth into 5 μm thick intervals, and then mean FWHM and standard deviation were calculated for each depth interval.

TABLE 1
system component list for the FIG. 2 embodiment

| Part | Description | Part Number (Vendor) |
|---|---|---|
| O1 | Olympus 60× water objective, 2 mm WD, 1.0 NA W | LUMPLFLN 60XW |
| | | (Olympus) |
| O2 | Mitutoyo 50× plano apo HR objective, 5.2 mm WD, | 58-237 (Edmund Optics) |
| | 0.75 NA | |
| TL1 | 38.1 mm EFL achromat, 12.7 mm dia. | 49-775-INK (Edmund |
| | | Optics) |
| SL1 | Plössl lens: two 50.8 mm EFL achromats, 12.7 mm | 49-777-INK (Edmund |
| | dia. | Optics) |
| Galvo | Galvanometer mirror with a 5-mm-diameter clear | 6210H (Cambridge) |
| | aperture | |
| SL2 | Plössl lens: two 101.6 mm EFL achromats, 25.4 mm | 49-783-INK (Edmund |
| | dia. | Optics) |
| TL2 | 76.2 mm EFL achromat, 25.4 mm dia. | 49-781-INK (Edmund |
| | | Optics) |
| Asph | 15 mm EFL aspheric collimator | F260FC-A (Thorlabs) |
| C0 | −15 mm EFL N-BK7 plano-concave cylindrical lens | LK1753L1-A (Thorlabs) |
| C1 | 50 mm EFL N-BK7 mounted plano-convex | LJ1695RM-A (Thorlabs) |
| | cylindrical lens | |
| C2 | 50 mm EFL N-BK7 mounted plano-convex | LJ1695RM-A (Thorlabs) |
| | cylindrical lens | |
| O3 | Nikon plan apo λ 20×/0.75 objective, 1 mm WD, | (Nikon) |
| | 0.75 NA | |
| TL3 | 35 mm EFL machine vision lens, f/1.4 | MVL35M1 (Thorlabs) |
| | 135 mm EFL, Sony Sonnar T*135 mm F1.8 ZA lens | SAL135F18Z (Sony) |
| Camera | Scientific CMOS camera | Zyla 4.2+ (Andor |
| | | Technology) |

FIG. 6 depicts an example of an imaging cap 82 that is fabricated to cover the imaging head (or, more specifically, the distal end of the first objective 12 of the imaging head). The cap 82 incorporates an optically transparent spacer to provide stabilization of the tissue being imaged. The cap 82 may be used together with either the FIG. 1 or FIG. 2 embodiments described herein. In some embodiments, the imaging cap 82 provides required water immersion as well as precision spacing of the primary objective (O1) from the tissue being imaged. It also advantageously immobilizes and stabilizes the tissue being imaged.

An example of a cap 82 was manufactured using 3D printing to fit over a standard objective lens 12. A circular glass coverslip 88 was glued to the front surface using cyanoacrylate glue and providing a water-tight seal. Once placed over the objective lens 12, water 85 was injected into the gap between the lens and the cap 82, coupling the objective 12 to the cover glass 88. Set screws (not shown) on the body of the cap 82 permitted fixation once the cap's position was adjusted to the right distance and alignment with the imaging plane. The external surface of the cover glass 88 was typically positioned at 50-150 microns from the primary focal plane of the objective 12 (e.g. 1.85 mm from the front surface of a 2 mm working distance objective). In this way, tissue pushed against the outer surface of the glass 88 could be imaged over a 300 micron depth range, if the focal plane is centered at a depth of 150 microns. (This distance choice could be made on the basis of the expected penetration depth into the tissue to be imaged.) In practice, the cap turned out to be extremely helpful for imaging unconstrained in vivo human tissues as it could be pressed against the tissue (e.g., oral tissue) to stabilize the tissue being imaged and the ability to slide across the tissue while maintaining the tissue at the desired working distance. In some embodiments, the cap may be sterilizable and/or disposable for patient protection.

The cap designed for use together with the FIG. 1 embodiment was shaped and dimensioned for attachment to a standard 60× 1.0 NA, 2 mm working distance, cover glass corrected, water immersion commercial objective lens (Olympus). This approach can be applied to any type of objective lens including miniaturized and custom lenses. For example, the lens could be fabricated to have a positioning groove or other guide for precise placement/attachment of the cap at the correct distance. The distance could also be adjustable by a mechanical, electrical, pneumatic or hydraulic mechanism. The use of a glass front surface 88 is ideal if the objective lens is cover-glass corrected, but alternative front surface media can be used if needed, including PTFE, as a refractive index match to water, or other materials such as PDMS or refractive index-specific polymers. In the latter cases, the entire spacer could be solid, or coupled to the objective lens with a small drop of water or refractive index matching medium. If the spacer is sufficiently rigid, the cap part extending back over the objective lens could be more supple, for example a thin plastic sheath with the spacer attached at the tip. In another embodiment, the spacer could be designed into the objective lens itself, providing a lens with ˜150 micron working distance. A thin, index-matched sheath could provide a disposable cover or the lens could be chemically or heat sterilizable. Optical components in the light path (including electrically tunable lenses) could be used to adjust the effective working distance of the objective to be able to adjust imaging depth range without needing to reposition the front surface.

Optionally, the cap can incorporate or accommodate ways to interact with the tissue such as to localize injection of a marker dye to the imaged location, or even to acquire a sample at the imaged location.

In Vivo, Label Free Human Imaging of the Oral Cavity with Medi-SCAPE

The cap configuration depicted in FIG. 6 was successfully used to acquire in vivo human data in the oral cavity of a healthy adult volunteer using both the FIG. 2 and the FIG. 1 embodiments. The caps were configured to ensure the optimal working distance for the respective objective to capture a 200-300 μm depth range into the tissue while maintaining a water immersion interface for the lens. In some embodiments the depth range is 50-350 μm.

Label-free roving scans of the tongue, inner and outer lip were acquired by asking the adult subject to position the appropriate tissue onto the imaging cap depicted in FIG. 6 and to slowly move its position during continuous volumetric imaging for up to 120 seconds at 3-5 VPS. These roving scans were stitched into a continuous, large 3D volume. Data from both the FIG. 1 and FIG. 2 embodiments consistently revealed features of the layers of the oral tissues including different types of tongue papillae and transitions between different tissue types that recapitulate standard features of histopathology of the oral mucosa. Bright fluorescence was visible on the tongue's filiform papillae likely from keratin and bacteria, while the fungiform papillae's epithelium was transparent, permitting an unobstructed view on the bright green inner branched structures that matched well with the structure of capillaries. Interestingly, one of the major sources of in vivo image contrast was found to be blood vessels, both from green autofluorescence of the vessel wall, and red signal corresponding to blood itself. In the lip, a diversity of different vascularized rete peg structures were seen progressing from fine and pointy in the inner lip to thicker and more stump-like at the transition from inner to outer lip. The transition from lip to skin captured striking features of hair follicles circled by microvasculature.

MediSCAPE's ability to image the regularity of patterns of these protrusions and the continuity of the basement membrane below the surface epithelium, as well as vascular patterns within the lamina propria suggests that MediSCAPE could feasibly detect a range of disorders of the oral mucosa, from ulceration and scar tissue to squamous cell carcinoma. Importantly, large areas of the tissue of interest were stitched to up to 13 mm for demonstration, can be canvassed for early detection of suspicious lesions and non-invasive follow-up and monitoring. The oral cavity was chosen for this first in-vivo human demonstration as it is readily accessible in healthy volunteers, however this data provides valuable evidence that MediSCAPE could be applied broadly to imaging in-vivo, in-situ human tissues in a wide range of clinical settings including dentistry, otolaryngology, ophthalmology, gynaecology and diverse open and laparoscopic surgeries and procedures.

Label-Free Volumetric Imaging of In Vivo Kidney and Heart in Real-Time

Although normally considered a nuisance in fluorescence imaging, autofluorescence in living tissue can allow visualization of morphological features routinely used for histological evaluation of tissues. Examples of intrinsic fluorescence sources in biological tissues include Elastin fibers, lipo-pigments (e.g., Lipofuscin and Ceroids), Phospholipids, and flavins (e.g., Flavin adenine dinucleotide, Riboflavin, and Flavin mononucleotide). These fluorophores are likely visible under 488 nm excitation in MediSCAPE. The ˜525 nm emission channel likely captures elastin, flavins, lipofuscin, ceroids, phospholipids, bilirubin and hyaline while the ˜618 nm channel captures relatively more signal from lipofuscin, ceroids and porphyrins.

Furthermore, the distribution and concentration of intrinsic fluorophores such as elastin and FAD provide a rich range of molecular information, which can indicate changes in tissue health even before structural changes become visible. Label-free imaging in humans is especially valuable because in vivo dye use is limited by safety restrictions and the complexity and cost of obtaining FDA-approval, limited penetration depth, heterogenous staining, and time sensitivity of dye administration in a clinical setting.

To demonstrate MediSCAPE's ability to capture in vivo autofluorescence contrast at high speeds, mirror-based scanning was used to image the exposed kidney and heart of a heavily anesthetized wild type mouse.

Benefits and Applications of MediSCAPE

MediSCAPE allows real-time volumetric imaging of intact, in vivo and fresh tissues without the need for exogenous dyes, which could allow simple but comprehensive assessment of tissues in a clinical setting. MediSCAPE's unique advantage over conventional confocal microendoscopy is its ultra-fast 3D imaging speed, combined with much higher sensitivity. These features permit high quality in vivo imaging of cellular features and 3D morphologies using only autofluorescence contrast, while tolerating in vivo motion and allowing dynamic surveillance of large areas of tissue in real-time. MediSCAPE can image a range of different exogenous fluorophores, extending its utility for broader clinical applications.

The primary clinical applications of MediSCAPE that we envisage are surgical guidance for lesion resection and biopsy site selection. The form factor of the FIG. 2 embodiment is currently compatible with open surgical fields including brain, heart, orthopedic and abdominal surgeries, tissues within accessible orifices such as the mouth and cervix, and potentially for laparoscopic and robotic surgeries. Results in a mouse model of pancreatic cancer suggest that MediSCAPE could provide valuable guidance during complex Whipple procedure surgery. A smaller form factor system, or GRIN lens-based extension of MediSCAPE could permit ‘probe’ type imaging that could guide or be incorporated into needle biopsy procedures.

MediSCAPE's ability to image intact tissues non-destructively could allow evaluation of tissue health, tissue-typing, nerve localization, mapping microvasculature and evaluation of reperfusion using intravascular dyes, for both clinical and veterinary applications. Furthermore, MediSCAPE's sensitivity to autofluorescence could be harnessed to reveal metabolic changes as novel disease biomarkers. MediSCAPE could also prove highly valuable in combination with wide-field imaging of targeted ‘molecular probes’ to visualize cellular-level uptake and disambiguate labeling, particularly during early clinical validation studies. As demonstrated by successful comprehensive imaging of fresh, resected tissues, MediSCAPE microscopy also has significant potential for rapid, 3D evaluation of biopsies and resected tissues at the bedside, with or without exogenous contrast agents.

Although MediSCAPE's penetration depth is limited by the scattering properties of the tissue being imaged, the high-speed 3D data produced is equivalent to 10-100's of sequential thin histology sections. In many situations this 3D information provides valuable additional information about tissue structures, while also permitting roving and stitching which would be impossible with 2D planar imaging. While this penetration depth limitation prevents non-invasive imaging of deep tissue structures, that the ability to image repeatedly during resection enables flexible interrogation of in-situ and residual margins as overlying tissue is removed. Penetration depth could also be improved with light sheet optimization, or use of red or near-infrared illumination, especially in concert with red-shifted contrast agents.

MediSCAPE also advantageously facilitates the rapid, non-destructive inspection of donor organs prior to transplantation. Many donor kidneys are discarded because of difficulties in evaluating their health within the short window of time between donation and transplantation. MediSCAPE's ability to visualize key diagnostic features in intact human kidney supports this potential application, which could extend to in situ evaluation and biopsy guidance in other transplant organs such as the liver and heart.

As demonstrated by the comprehensive imaging of fresh, resected tissues with stage-scanned acquisition, MediSCAPE microscopy also has significant potential for rapid, 3D evaluation of biopsies and resected tissues at the bedside. MediSCAPE far surpasses the 3D imaging speed limitations of point-scanning confocal, two-photon and Raman microscopy approaches while avoiding the need for costly specialized lasers that can be challenging to locate at the bedside. Moreover, since imaging excised tissues removes constraints on utilizing a wide range of fresh-tissue compatible selective dyes and labels, MediSCAPE results in stained fresh tissues show that bedside forms of MediSCAPE could provide a more comprehensive evaluation of biopsied tissue as a complement/cross validation to its in vivo use. Ex vivo tissues can also be chemically cleared to provide a more comprehensive 3D visualization. Although tissue clearing steps can take excessive time, cleared tissues can also be imaged using the FIG. 1 embodiment, offering advantages over dual-objective light sheet systems including simplicity of the single objective light-sheet geometry and the ability to image to the full depth of the primary objective's working distance.

Sources of Contrast

The majority of images described herein were acquired with a single 488 nm laser for fluorescence excitation. However, a wider range of excitation wavelengths could readily be incorporated into MediSCAPE, including 405 nm, 561 nm and near infrared ranges. Additional wavelengths could harness autofluorescent molecules such as NADH, collagen or retinol, as well as exogenous dyes extending into the near infrared such as indocyanine green.

Although we compared autofluorescence imaging to conventional histological contrast as a gold standard, autofluorescence has the potential to reveal additional valuable information beyond what is seen in histology. For example, the autofluorescence detected with 488 nm excitation in the human kidney was particularly strong in the elastic lamina of arterial walls, cytoplasmic lipofuscin deposits and urinary cast material. Also clearly visible were cytoplasmic granular structures within epithelial cells of pseudo-hypertrophied proximal tubules and focal arterioles with intense punctate perinuclear autofluorescence suggestive of lysosomal signals. Nearly all of these tissue features appear much less distinctive in routine histology, suggesting the potential for MediSCAPE to gather adjunctive information beyond what traditional histology can provide. New diagnostic features could have great clinical significance, particularly for limited or rare human tissue specimens such as small needle core biopsies.

Visualization, Display and Automated Analysis

A key factor in clinical adoption of MediSCAPE will be the way in which data can be visualized and interpreted in real-time by both the acquiring surgeon and the examining pathologist. All analysis and rendering of MediSCAPE images described herein were performed offline, however real-time stitching and visualization of depth and lateral cross-sections should be feasible using field programmable gate array (FPGA) technologies that work well for real-time visualization and rendering of ultrasound and OCT data. Moreover, the digital nature of MediSCAPE's data would permit online inspection of datasets by remote pathologists (as is common in radiology) who could readily select their preferred views and color schemes. MediSCAPE's rich volumetric data is also ideally suited for automated machine learning-based analysis that could automatically classify normal and suspicious areas and pick out key tissue features. Online analysis results could be projected onto the surgical field of visualized using augmented reality. Where available, MediSCAPE data could be spatially registered to stereotactic coordinates and other imaging modalities like Mill, and fully archived as part of the patient's electronic health records.

Technological Development and Form Factor

Although most of the results described herein utilized the FIG. 1 embodiment of MediSCAPE, we also presented near equivalent performance with the FIG. 2 embodiment, whose form factor is compatible with being mounted on a surgical microscope frame and hand-guided within the surgical field. Further miniaturization using MEMs mirrors, fiber optics or rod and grin lenses, and custom-built small diameter, high NA objectives could all further reduce the form factor of the system to permit laparoscopic and even endoscopic use.

For routine clinical use, the system optionally uses an optically transparent spacer at the tip of the primary objective to press against tissue at the optimal working distance, incorporated into a disposable or sterilizable sheath. Features such as microscale stabilization and auto-scanning over fixed distances could improve ease of use, while the ability to mark, capture or even laser-ablate identified regions in concert with imaging could provide significant benefits for microscale resection. MediSCAPE's ability to dynamically zoom into features of interest is also beneficial, providing a compromise between covering larger areas via roving and capturing key features of disease in the tissue of interest.

In summary, MediSCAPE is a powerful new approach to in situ histopathology, leveraging the unique benefits of light-sheet scanning to allow high-speed 3D, label-free imaging of a wide range of tissues. MediSCAPE has the potential to go beyond replacing biopsies and conventional histopathology, opening new doors to non-destructive assessment of a wide range of valuable tissue features in situ. These new capabilities could greatly improve standard of care while also reducing the time and cost of a wide range of surgical procedures.

Data Processing

MediSCAPE data processing consisted of background subtraction, skew-correction of data and merging dual color images with a custom-written MATLAB graphical user interface (GUI). A pseudo-flatfield correction was applied to dual color images along the x- and y-axes by dividing volumes by a Gaussian-blurred mean intensity z projection. For better visualization of details, MediSCAPE data shown in Image Set 3 was processed with an unsharp mask (radius 1, amount 0.3) and CLAHE histogram equalization (block size, 75, slope 2).

For H&E pseudocoloring of MediSCAPE datasets acquired with proflavine staining, the virtual H&E algorithm developed by Giacomelli et al was used to create brightfield H&E color channels from fluorescence data based on Beer-Lambert's law. Autofluorescence emission collected through a 618/45 nm bandpass filter with 488 nm excitation was used to indicate general non-nuclear background structure on a log-scale (eosin) while proflavine fluorescence excited at 488 nm was used to indicate nuclear structure (hematoxylin).

Data Stitching

A key feature of MediSCAPE is its very fast 3D imaging speed, even when imaging weak autofluorescence. This speed can be leveraged to allow exploration of large areas of tissue by ‘roving’ or continuously moving the tissue relative to the system's 3D field of view. MediSCAPE's speed can tolerate this translation without significant artifacts in each individual volume, and since each volume has some spatial overlap with the last, a sequence of volumes can be stitched to generate a fully contiguous 3D strip of data spanning millimetres or more. This feature does not require continuous or motor-controlled movement and can tolerate unavoidable in vivo movements such as breathing, making it ideal for evaluating transitions between tissue types, or exploring heterogenous regions for multi-scale spatial patterns at the cellular and mesoscopic level.

To stitch consecutively-acquired overlapping volumes from a roving scan, a custom ImageJ macro was written using the existing Pairwise Stitching plugin in Fiji to act on volumes already saved as background-subtracted, dual color tiff-stacks in MATLAB. Volumes were stitched pairwise to simulate real-time stitching which could be implemented using an FPGA. Because the volume rate was generally much higher than the speed at which tissue was translated during acquisition, every nth volume (where n=2-5) acquired was used for stitching to decrease total processing time and reduce stitching errors. Stitched volumes shown in Image Set 1, Image Set 2, Image Set 7, and Movies 1, 3, and 10 (described below) were created by fusing approximately every 4th consecutively-acquired volume in a pairwise fashion. During each stitching step, volumes were downsampled 2× along the Y and Z axes and aligned coarsely given the alignment positions found in the previous successful stitching step. If the alignment r-value was over a given threshold (˜0.8), fine alignment of the raw volumes was performed using the initial coarse alignment values and raw volumes were fused using linear blending with a 10% overlap. If the coarse alignment r-value was under the threshold due to excessive motion, the next consecutive volume was loaded and aligned and so on until both volumes could be aligned accurately. Data was skew-corrected in MATLAB after stitching. To create the “real-time stitching” movies described below for Movie 1 and Movie 3, each stitching step was skew-corrected and positioned on a blank canvas the same 3D size as the final fully stitched volume.

The Bigstitcher plugin in Fiji was used to stitch stage-scanned data. A custom MATLAB and ImageJ pipeline was implemented to automatically save background-subtracted, skew-corrected dual color tiff stacks in MATLAB, convert and load data in HDFS format into BigStitcher, pre-align volumes with stage coordinates and stitch data using linear blending with default stitching wizard presets and fine ICP alignment.

Fourteen sets of sample images (referred to herein as Image Set 1-Image Set 14) and ten movies (referred to herein as Movie 1-Movie 10) were captured/made to demonstrate the capabilities of the hardware described herein. More specifically:

Image Set 1 showed a volume rendering and individual planes of the in vivo mouse kidney collected as 802× 861×275 μm³ dual color xyz volumes at a sampling density of 1× 1.4× 1.1 μm³/voxel in 0.78 sec with mirror-based scanning. Autofluorescence was excited with 488 nm light and dual emission channels were collected through 525/45 nm and 618/45 nm bandpass filters, using blue and “yellow hot” colormaps which allowed better visualization of the overlapping channels.

Tubules showed robust autofluorescence in both emission channels, with proximal tubules showing higher emission at ˜525 nm (yellow hot) than distal tubules (blue/purple). Autofluorescence in this range is likely due to flavins in the metabolically active proximal tubule cells. Nuclei could be distinguished along the tubule walls as punctate dark regions. H&E histology processed from a similar region of a mouse kidney cortex showed normal tubular architecture. Structural information between both types of images was similar but MediSCAPE provided additional molecular contrast based on spectrally-resolved emission of endogenous fluorophores, including flavins, elastin, porphyrins and lipofuscin.

A key feature of MediSCAPE is that this autofluorescence contrast can be captured in real-time, allowing easier exploration of large 3D fields of view in tissue. To capture a ‘roving scan’, the anesthetized mouse was manually translated along 3 dimensions to mimic how a MediSCAPE imaging probe would rove over intact, in vivo tissue. Dual color volumes of 358×798×165 μm³ in xyz, with a sampling density of 2.5×1.4×1.1 μm³/voxel, were acquired at 9.3 VPS while continually roving across the intact kidney cortex surface. In addition to providing a continuous sequence of high-quality volumetric images across a 1×3 mm strip of living kidney, this roving data was stitched to generate a contiguous volume. To generate this larger field of view, overlapping 3D volumes were stitched using the pairwise stitching plugin in ImageJ, similar to how volumes would be stitched in real-time on a field programmable array (FPGA). In a more detailed image, nuclei appeared as negative space along tubule walls with clear differentiation between proximal and distal tubules based on both structure and spectral emission. These features were similar to a previous single volume scan, despite coarser 2.5 μm x-steps over a reduced x-range and depth range, allowing for real-time speeds. Movie 1 (described below) showed real-time playback of the roving scan with lateral and depth cross-sections from each volume acquired and stitching of a larger field of view as overlapping volumes are acquired.

Although cellular features can be seen at this sampling density, MediSCAPE has the ability to trade-off resolution with field of view to ‘zoom in’ on features of interest. Movie 2 (described below) showed mouse kidney data acquired using a variable 70-200 mm focal length tube lens, switching between regular (4.6×) and high magnification (11.4×), revealing crisper and more detailed visualization of tubular structure. This ‘zoom in’ feature could be readily automated and co-stitched with coarser imaging over larger fields of view acquired at lower magnification.

Image set 2 demonstrated MediSCAPE's tolerance to inherent in vivo motion. The same in vivo preparation was used to image the beating, intact, in vivo mouse heart. Data was acquired by roving across the exposed heart surface while continuously acquiring dual color volumes at 12.9 VPS (galvanometer-scanning over a volume size of 305×798×138 μm³ with a sampling density of 2.5×1.4×1.1 μm³/voxel). Image set 2 showed xy slices within a 3D stitched field of view created from 15.6 sec of data acquired while using the 3-axis stage to manually rove over cardiac tissue. Striated cardiac muscle cells in the myocardium were clearly visualized. Veins and arteriesappeared as negative space, although arteries could be distinguished by the highly autofluorescent elastin along their walls. Elastic fibers could also be seen on the surface of the myocardium. Granular autofluorescence along the muscle fibers is likely lipofuscin, a lipopigment which accumulates in highly active cells over time. Periodic cardiac pulses that occurred during this acquisition appeared as sudden lateral movements along the y-axis in a kymograph, where a maximum intensity projection of x and z was shown over 15.6 seconds of imaging. The system was able to acquire volumes which could be successfully stitched together, with minimal visible motion artifact or blur. Movie 3 (described below) showed real-time playback of the cross-sections of the beating heart as well as stitching of these volumes as they were acquired. Note that stitching three-dimensional tissue volumes compensates for tissue motion in all 3 dimensions and allows roving laterally and along the depth axis. Compared to stitching traditional 2D fields of view, volume stitching more reliably reconstructs inherently 3D tissue structures, correcting for out-of-plane motions which are unavoidable in vivo.

Image Set 3 demonstrated characterization of tissue structures visible with solely autofluorescence in a wide variety of freshly excised mouse tissues imaged with MediSCAPE. Image Set 3 showed xy lateral slices at various tissue depths with H&E histology showing the same or adjacent regions in the mouse tissue. Movies 3 and 4 (described below) showed complete depth fly-through movies of each 3D volume. The freshly excised tissues included the following: cardiac muscle fibers in the heart ventricle, cerebellum in a sagittally cut surface of the brain, alveoli and visceral pleura in the lung, classic hepatocyte cord formations and capsule in a liver lobule, red pulp and the surrounding capsule in the spleen, superficial layers in the bladder mucosa with pixel intensities shown on a log scale for better visualization, muscle fibers visible deep within thigh muscle, and crypts of Lieberkühn in the colon mucosa.

With solely intrinsic contrast, micron-scale structures were visible in all fresh tissues studied, and corresponded well to structures visible in H&E histology. The shape and diameter of crypts of Lieberkühn, for instance, could be clearly distinguished in the colon mucosa. Alveoli in lung tissue were distinctly lined with intensely fluorescent elastin could be assessed intact whereas histology often showed major distortions from sectioning of the delicate air-filled tissue. Layers within tissues, like the bladder mucosa were distinct and could be assessed in 3D, allowing more comprehensive evaluation than 2D histology sections and single plane confocal microendoscopy. The furthest depth at which histology-level resolution is possible is tissue dependent, and also varies with the excitation wavelength. For many tissues, resolution starts to degrade after 50 μm with 488 nm excitation. Yet in skeletal muscle, for example, cellular-level contrast was observed 121 μm into the tissue. Images were acquired at a volume size of 801×1065×275-330 μm³ in xyz, with a sampling density of 1×1.4×1.1 μm³/pixel, respectively, at 100 fps with 5-7 mW of laser power at the sample.

Detection of Histologic Features Associated with Disease States in Human Tissue

To test MediSCAPE's ability to capture disease-related features in human tissues, fresh human kidney tissue was obtained from surgical nephrectomy specimens and imaging results were compared to conventional periodic acid-Schiff (PAS) and H&E histology on the same samples.

Image Set 4 showed autofluorescence imaged by MediSCAPE in a nephrectomy specimen from a patient with underlying chronic kidney disease (CKD). To co-locate regions in SCAPE images with those in histology images, we imaged the entire flat face of the fresh specimen using motorized stage-scanning to acquire and stitch a 13.3×10.6×0.3 mm³ volume. Imaging data was acquired in 196 seconds. Movie 7 (described below) showed a depth fly-through movie showing lateral cross-sections in the full stitched volume. From the full stitched volume, a 2.1×1.6 mm² xy ROI was obtained.

Examples of key diagnostic features identified by MediSCAPE included arteriosclerosis and arteriolar hyalinosis. Identification of arteries was aided by strong autofluorescence of the internal elastic lamina of arterial walls, even more prominent on MediSCAPE imaging in the setting of hypertensive arteriosclerosis in which there is luminal narrowing by intimal thickening with reduplication of the elastic lamina. We could clearly identify glomeruli and distinguish those showing global sclerosis. We could also discriminate sub-glomerular structural elements including the glomerular capillary tuft, Bowman's space and Bowman's capsule, especially when the latter had undergone partial sclerosis (Image Set 4d, note arrows). In addition, we could identify several glomerular features related to CKD including segmental glomerulosclerosis, focal hyalinosis, and nodular mesangial sclerosis (data not shown). In the tubulointerstitial compartment, characteristic chronic changes including tubular atrophy and interstitial fibrosis, known to have the strongest correlation with renal outcomes, were clearly evident in MediSCAPE images. We could distinguish atrophic from non-atrophic tubules, identify pseudo-hypertrophy of proximal tubules and tubular casts.

Image Set 5 highlighted the unique value of MediSCAPE's isotropic 3D imaging of intact, fresh tissues. Image Set 5 showed an example of a clinically relevant lesion that can be ambiguous to identify from 2-dimensional thin sections. In several planar images, a small cyst-like structure was evident, which on a single image could be either a severely dilated atrophic tubule or a simple renal cyst. However, the 3D data revealed a residual compressed and sclerosed capillary tuft, pressed against the internal wall of the cyst-like space, distinguishing this structure as an atubular glomerulus (or “glomerular microcyst”) rather than any type of tubular-derived element. Examining the perirenal fat from normal human kidney tissue, we also found that MediSCAPE could also capture the 3D arrangement of elastic fibers and adipocytes on the basis of their intrinsic autofluorescence. Evaluating these features in volumetric space could allow more accurate assessment of fat content, as well as the structure, density, and identity (eg. elastic versus collagen) of fibers in different tissue compartments.

Volumetric Imaging of Topical Dyes in Fresh Human Tissue

Image Set 6 demonstrated that MediSCAPE can image clinically relevant features using a wide range of fluorescent contrast agents, if available. Image Set 6 showed an example of MediSCAPE data collected from a sample of fresh, normal human kidney stained with proflavine, a topical nuclear dye commonly used in clinical imaging research. Proflavine and red autofluorescence emission was acquired with 488 nm excitation using stage-scanning to create a 7500×918×164 μm volume in 5.6 seconds. Proflavine staining revealed nuclear size, shape and distribution, while autofluorescence provided complementary structural information. Following recent conventions for visualization of tissue fluorescence in histopathology, we also generated dual color MediSCAPE data with a pseudocolor H&E colorscale, using proflavine as a hematoxylin analogue (purple) and with “eosin” represented by the autofluorescence signal collected with a 618/45 nm bandpass filter (pink). Pseudocolored MediSCAPE images closely resemble conventional brightfield H&E histology could allow easier evaluation of nuclear detail when needed. Movie 8 (described below) showed a fly-through of the top 30 μm in depth of the pseudocolored MediSCAPE volume.

Roving In Vivo Volumetric Imaging of Perfusion Using Intravascular Fluorophores

Image Set 7 demonstrated MediSCAPE's ability to perform real-time 3D in vivo imaging of microvascular perfusion. We imaged the brain of a living, head-fixed mouse through a glass cranial window following intravenous injection of dextran conjugated fluorescein. Roving scans were acquired at 9 VPS, and a 3D stitched volume and multi-view maximum intensity projections of single volumes were generated. Movie 10 (described below) showed the real-time roving data showing clear ability to observe dynamic flow in the vessels, while also capturing clear details of the 3D microvascular architecture without motion artifacts. In addition to neurological surgery applications, this approach could be of value to assess microvasculature in tumor margins or after tumor embolization, arteriovenous malformations and organ reperfusion. MediSCAPE could leverage commonly used intravascular fluorophores such as fluorescein and the near infrared fluorophore indocyanine green for deeper tissue penetration.

Image Set 8 demonstrated MediSCAPE images of 200-nm fluorescent beads embedded in gel. Maximum intensity projections over 60 μm ranges were taken along all three axes from skew-corrected raw data. Each cross-section was scaled to give an isotropic um/pixel over an xyz field of view of 390×742×145 μm³.

To compare the FIG. 2 system's imaging performance to the FIG. 1 system, we imaged fresh, unstained mouse tissues. Using 488 nm excitation and ˜4.6 mW incident power on the sample, we used galvanometer scanning to collect dual color volumes of size 400×700×162 μm³ in xyz, acquired with a sampling density of 1.0×1.4×1.08 μm³/voxel in xyz. Images were collected at 300 Hz (0.75 VPS) to compare tissue structures to those acquired with the FIG. 1 system. Image Set 10 showed cross-sections displaying tubules in the kidney cortex, the fibrous capsule and underlying cords of hepatocytes and sinusoids in the liver, cardiac muscle in the surface of the heart, and crypts of Lieberkühn in the colon mucosa. These volumes demonstrate very similar penetration depth and resolution of tissue structures compared to the FIG. 1 system albeit with a smaller, rounded field of view (caused by the use of a smaller form-factor 60× objective as O1).

As a further demonstration of imaging performance in fresh tissue, we acquired 250×700×136 μm³xyz volumes continuously at 11.2 VPS while roving across the colon mucosa. A stitched field of view from 16 seconds of roving was created by pairwise stitching approximately every other volume collected. Clear crypt structure was visible along each dimension, even with 2 μm sampling density along x and frames collected at 1400 fps.

During these scans, equivalent signal levels were detected on the Andor Zyla 4.2+camera. The well-matched signal to noise and resolution of both systems are consistent with models predicting that the FIG. 2 form of MediSCAPE actually collects a larger NA of emitted light, and is thus even more light-efficient than the FIG. 1 system. The primary trade-off for the smaller form factor is the size of the field of view, resulting from the use of a more compact 60× primary objective lens in the FIG. 2 embodiment. This demonstration supports the feasibility of implementing MediSCAPE even through smaller primary objective lenses and longer, narrower telescopes or relay lenses to permit simple maneuvering and precise positioning of the MediSCAPE imaging head into the surgical field.

Image Set 10 showed Label-free imaging of fresh mouse tissue with the FIG. 2 embodiment. xy (top) and yz (bottom) cross-sections in various fresh mouse tissue were acquired with the FIG. 2 system with 488 nm excitation and dual color emission channels. Cross-sections showed tubules in the kidney cortex, capsule and underlying cords of hepatocytes in the liver, cardiac muscle in the heart and crypts of Lieberkühn in the colon mucosa. Image quality was similar to the FIG. 1 design, with nuclei visible in kidney tubules, crypts of Lieberkühn in the colon mucosa and individual elastin fibers in the liver capsule. The primary difference is a reduced field of view, which may be mitigated by stitching larger fields of view by roving across tissues.

Image Set 10 demonstrated dual color autofluorescence visualization. xy image planes were acquired by MediSCAPE in fresh mouse brain cortex, including a prominent blood vessel. Contrast corresponded to autofluorescence excited by 488 nm light. Dual color emission images were acquired simultaneously using an image splitter in front of the camera and positioning each color channels side-by-side on the camera chip (along y). Two images in this set showed grayscale raw emission channels acquired with 525/45 nm and 618/45 nm bandpass filters respectively. These channels were converted to ‘yellow hot’ and blue colormaps and then merged into another image in the set.

Image Set 11 demonstrated Autofluorescence in diabetic human kidney tissue imaged with MediSCAPE. Autofluorescence captured by MediSCAPE revealed features common to and beyond those seen on routine histology. One image in this set was a PAS histology image of kidney cortex tissue from an older, diabetic patient with features of mild diabetic nephropathy. Another image in this set was a MediSCAPE xy slice from a stage-scanned volume of the same piece of tissue (while fresh) showing autofluorescence excited at 488nm. Kidney capsule and urinary casts were seen in both MediSCAPE and PAS images. Another image in this set showed a focal subcapsular collection of tubules with autofluorescent cytoplasmic granules. Urinary cast material was also evident in the xy plane, and further evidenced by characteristically strong autofluorescence in the yx plane. Another image in this set showed tubules with accentuated peritubular autofluorescence. Another image in this set showed glomerulus with focal autofluorescent granules.

Image Set 12 demonstrated MediSCAPE label-free imaging of elastic fibers and fat cells in human perirenal fat. One image in this set was a 3D rendering (ImageJ 3DViewer) of a section of normal human perirenal fat showing highly fluorescent elastic fibers and fat cells. Another image in this set was a yz cross-section from the plane indicated shows layering of fibers over fat cells, which could be distinguished as circular yellow droplets. Another image in this set was a lateral cross-sections in which fat cells and intersecting vessels were visible.

Image Set 12 also demonstrated stained human kidney tissues imaged with MediSCAPE. Fresh human kidney tissues showing features of arterionephrosclerosis were stained with nuclear dye, either methylene blue or proflavine, imaged by MediSCAPE, then processed for histology where the same tissue block faces were stained with PAS and/or H&E. Three images in this set demonstrated how the 4 main renal histologic components which must be routinely evaluated with both PAS and H&E histology appear in PAS histology, an xy slice of a MediSCAPE volume stained with methylene blue and H&E histology. These images revealed glomeruli, arteries, tubules, and interstitium. Methylene blue in the MediSCAPE image defined cellular cytoplasmic, nuclear and extracellular compartments, similar to H&E but better highlighted arterial elastic lamina and tubular and interstitial compartments, similar to PAS histologic sections.

A second biopsy piece from the same patient showed scarred tubulointerstitium in a focal area of fibrosis in both the MediSCAPE and corresponding H&E histology images. Another image in this set was a 3D rendering (Imaris) of the larger stage-scanned volume acquired on MediSCAPE. This image showed the 3D structure of fibrosis, arteries and glomeruli. The origin of the xz depth section was visible. Two more images in this set showed a non-sclerotic glomerulus in more detail across 20um in depth.

All 4 renal histologic compartments commonly assessed through a combination of H&E and PAS histology could be clearly distinguished in MediSCAPE images, especially with methylene blue staining.

Image Set 14 demonstrated a comparison of topical dyes applied to fresh mouse colon mucosa. Single xy and yz slices in samples of fresh mouse colon mucosa were imaged with MediSCAPE. In three images from this set, contrast was derived from a) 0.01% proflavine, a nuclear dye (exc. 488 nm, em. 525/45 nm), b) 1% methylene blue, a clinically-used nuclear dye (exc. 637 nm, em. >685 nm), and c) fluorescein sodium, an FDA-approved topical and IV dye (exc. 488 nm, em. 525/45 nm). Locations of corresponding cross-sections and nuclei were visible. Depth penetration of topically applied dyes was both stain- and tissue-dependent, as seen in yz depth sections. Crypts of Lieberkühn and goblet cells were visible. These results demonstrated MediSCAPE's ability to capture a variety of exogenous contrasts with high signal to noise, and also highlighted the challenges of ensuring dye penetration compared to exploiting intrinsic contrast.

Movie 1 demonstrated Label-free in vivo mouse kidney imaged with MediSCAPE at 9.3 VPS. xy and yz cross-sections from 358×798×165 μm³ dual color volumes were collected at 9.3 VPS while roving across an in vivo mouse kidney. A 3D rendering (ImageJ 3D Viewer) and a lateral cross-section of a larger field of view were stitched from overlapping volumes as they were collected. Playback was in real-time with stitching of volumes done in post-processing. Imaging parameters were as shown below in Table 2.

Movie 2 demonstrated Fresh mouse kidney imaged with low and high magnification on MediSCAPE. Autofluorescence in the same region of fresh kidney tissue was imaged at different magnifications. A variable focal length tube lens allows dual color imaging at a range of magnifications, adjustable based on the resolution, field of view and volume speeds required. One set of data was collected with the variable tube lens set to f=70 mm for 4.6× magnification while another set of data was collected in the same region, immediately after setting the variable tube lens to f=170 mm for 11.4× magnification. Autofluorescence emission in the —525 nm range was shown in yellow hot, while emission at ˜618 nm is shown was blue. Imaging parameters were as shown below in Table 2.

Movie 3 demonstrated Label-free MediSCAPE imaging of in vivo mouse heart imaged at 12.9 VPS. xy and yz cross-sections from 305×798×138 μm³ dual color volumes were collected at 12.9 VPS while roving across an intact, beating mouse heart. Cardiac pulses could be seen periodically in individual volumes and in the maximum intensity projection of data over time. Lateral (xy) cross-sections at different z-depths from a larger 3D field of view were stitched from overlapping volumes as they were collected. Playback was in real-time with stitching of volumes done in post-processing. Autofluorescence emission in the —525nm range was shown in yellow hot, while emission at ˜618nm was shown in blue. Imaging parameters were as shown below in Table 2.

Movie 4 demonstrated Autofluorescence in fresh mouse heart, brain, lung and liver imaged with MediSCAPE. Depth flythroughs of intrinsic contrast were imaged in the first 50 μm of intact, freshly excised mouse heart, sagittally cut cerebellum, intact lung and intact liver tissue. Autofluorescence emission in the ˜525nm range was shown in yellow hot, while emission at ˜618nm was shown in blue. Imaging parameters were as shown below in Table 2.

Movie 5 demonstrated Autofluorescence in fresh mouse spleen, bladder, muscle and colon imaged with MediSCAPE. Depth flythroughs of intrinsic contrast were imaged in the first 100 μm of intact, freshly excised spleen surface, bladder mucosa, thigh muscle and colon mucosa. Autofluorescence emission in the ˜525 nm was shown in yellow hot, while emission at ˜618 nm was shown in blue. Imaging parameters were as shown below in Table 2.

Movie 6 demonstrated Fresh mouse kidney, liver, heart and colon imaged with the FIG. 2 system. Depth fly-throughs of intrinsic contrast were imaged in the first 50 μm of four types of fresh, intact mouse kidney, liver, heart and colon mucosa. Autofluorescence emission in the ˜525 nm range was shown in yellow hot, while emission at ˜618 nm was shown in blue. Imaging parameters were as shown below in Table 2.

Movie 7 demonstrated MediSCAPE autofluorescence image of fresh human kidney biopsy with chronic kidney disease. A 13.3×10.6×0.3 mm xyz field of view was stitched from 12 stage-scanned dual color volumes acquired in a total of 196 sec. Autofluorescence emission in the ˜525 nm range was shown in yellow hot, while emission at ˜618 nm was shown in blue. Imaging parameters were as shown below in Table 2.

Movie 8 demonstrated Depth fly-through of H&E pseudocolor MediSCAPE images of fresh, normal human kidney tissue stained with proflavin. Proflavine fluorescence was encoded as hematoxylin (purple) and red autofluorescence emission encoded as eosin (pink) (488 nm excitation). A full 7500×918×164 um xyz volume was acquired by stage scanning in 5.6 s.

Movie 9 demonstrated A 3D rendering and fly-through of proflavine-stained human kidney tissue imaged with MediSCAPE. A 2732×921×273 um xyz stage-scanned volume showed signs of arterionephrosclerosis. A focal area of cortical scarring with tubular atrophy and interstitial fibrosis was evident near the center. Glomeruli and arteries were clearly visible by autofluorescence and proflavine signals excited at 488nm, and their structures could be more easily evaluated by scrolling through both lateral and depth cross-sections.

Movie 10 demonstrated Roving MediSCAPE imaging of in-vivo mouse brain vasculature with IV FITC-dextran. Volumes were acquired through a glass cranial window at 9 VPS while roving around the cranial window. 3D rendering of real-time data was performed during roving. A larger 3D field of view was built up by stitching overlapping volumes as they were collected. Playback was in real-time with stitching of volumes done in post-processing.

TABLE 2
Imaging Parameters for MediSCAPE data.

| | | | | | | Volume | | Laser |
|---|---|---|---|---|---|---|---|---|
| | | | | xyz | | Rate OR | | Power |
| Image | | | | Sampling | Frame | Total | | @ |
| Set/ | | | xyz FOV | Density | Rate | acq | | Sample |
| Movie # | Panel/Sample ^{a} | Scan Type | (um³) ^{b} | (um³/vox) ^{c} | (Hz) | Time ^{d} | | (mW) ^{e} |
| Image 1, | b, c, d; in vivo mouse | 1. mirror, | 802 × 861 × 275 | 1 × 1.4 × | 1021 | 0.79 | s | 7.5 |
| Movie 1 | kidney | stationary | | 1.1 | | | | |
| | h, i; in vivo mouse kidney | 2. mirror, roving | single vol: 358 × | 2.5 × 1.4 × | 1335 | 9.3 | VPS | 7.5 |
| | | | 798 × 165 | 1.1 | | | | |
| | | | stitched: 2813 × | | | | | |
| | | | 872 × 178 | | | | | |
| Image 2, | a, b; in vivo mouse heart | 2. mirror, roving | single vol: 305 × | 2.5 × 1.4 × | 1578 | 12.9 | VPS | 7.5 |
| Movie 3 | | | 798 × 138 | 1.1 | | | | |
| | | | stitched: 4830 × | | | | | |
| | | | 1102 × 185 | | | | | |
| Image 3, | a, c-g; mouse heart, lung, | 1. mirror, | 801 × 1065 × 330 | 1 × 1.4 × | 100 | 8 | s | 5 |
| Movie 4, | liver, spleen, bladder, | stationary | | 1.1 | | | | |
| 5 | muscle | | | | | | | |
| | b, h; mouse brain, colon | 1. mirror, | 802 × 1065 × 275 | 1 × 1.4 × | 100 | 8 | s | 7.5 |
| | | stationary | | 1.1 | | | | |
| Image 4, | Patient #1 (CKD), | 3. stage-scan | 12 strips: 13400 × | 1 × 1.4 × | 814 | 196 | s | 7.5 |
| 5 | human kidney cortex tissue | | 984 × 275, | 1.1 | | | | |
| Movie 7 | | | stitched: 13301 × | | | | | |
| | | | 10629 × 309 | | | | | |
| Image 6, | Patient #2 (multicystic | 3. stage-scan | 7500 × 918 × 164 | 1 × 1.4 × | 1335 | 5.6 | s | 7.5 |
| Movie 8 | kidney), | | | 1.1 | | | | |
| | proflavine, normal cortex | | | | | | | |
| | tissue | | | | | | | |
| Image 7, | FITC-dextran, | 2. mirror, roving | single vol: 305 × | 2 × 1.21 × | 1370 | 9 | VPS | 0.5 |
| Movie 10 | in vivo mouse brain | | 756 × 159 | 1.06 | | | | |
| | | | stitched: 1288 × | | | | | |
| | | | 1880 × 258 | | | | | |
| Movie 6 | a-d; mouse kidney, liver, | 1. mirror, | 400 × 700 × 162 | 1 × 1.4 × | 300 | 1.3 | s | 4.6 |
| | heart, colon | stationary | | 1.08 | | | | |
| | e; mouse colon | 2. mirror, roving | single vol: 250 × | 2 × 1.4 × | 1407 | 11.2 | VPS | 4.6 |
| | | | 700 × 135 | 1.08 | | | | |
| | | | stitched: 4130 × | | | | | |
| | | | 834 × 170 | | | | | |
| Image 8 | mouse brain | 1. mirror, | 803 × 1061 × 275 | 1 × 1.4 × | 200 | 4 | s | 7.5 |
| | | stationary | | 1.1 | | | | |
| Image 12 | Patient #3 (diabetic, | 3. stage-scan | 4206 × 970 × 275 | 1 × 1.4 × | 496 | 8.5 | s | 5 |
| | hypertensive) | | | 1.1 | | | | |
| | kidney cortex tissue | | | | | | | |
| Image 13 | Patient #2 (multicystic | 1. mirror, | 800 × 966 × 220 | 1 × 1.4 × | 200 | 4 | s | 7.5 |
| | kidney), | stationary | | 1.1 | | | | |
| | perirenal fat tissue | | | | | | | |
| Image 14, | a-c; Patient #4 | 3. stage-scan | 4110 × 1121 × 274 | 1 × 1.4 × | 407 | 10.1 | s | 0.25 |
| Movie 9 | (lumpectomy), | | | 1.1 | | | | |
| | proflavine normal tissue | | | | | | | |
| | #1 | | | | | | | |
| | d-h; Patient #4 | 3. stage-scan | 2732 × 1121 × 274 | 1 × 1.4 × | 407 | 6.7 | s | 0.25 |
| | (lumpectomy), proflavine | | | 1.1 | | | | |
| | normal tissue #1 | | | | | | | |
| | i-k; Patient #4 | 1. mirror, | 800 × 1121 × 165 | 1 × 1.4 × | 777 | 1 | s | 1 (637 |
| | (lumpectomy), | stationary | | 1.1 | | | | nm) |
| | methylene blue normal | | | | | | | |
| | tissue #2 | | | | | | | |
| Image 8 | a, proflavine, mouse colon | 1. mirror, | 800 × 1120 × 165 | 1 × 1.4 × | 755 | 1 | s | 0.1 |
| | | stationary | | 1.1 | | | | |
| | b, methylene blue, mouse | 1. mirror, | 800 × 971 × 165 | 1 × 1.4 × | 789 | 1 | s | 7.5 (637 |
| | colon | stationary | | 1.1 | | | | nm), |
| | c, fluorescein, mouse colon | | | | | | | 0.2 (488 |
| | | | | | | | | nm) |
| | d, mouse colon | 1. mirror, | 801 × 1103 × 275 | 1 × 1.4 × | 200 | 4 | s | 7.5 |
| | | stationary | | 1.1 | | | | |
| Movie 2 | mouse kidney (left, low | 3. stage-scan | 500 × 436 × 275 | 1 × 1.4 × | 496 | 16 | s | 7.5 |
| | mag) | | | 1.1 | | | | |
| | mouse kidney (right, high | 3. stage-scan | 500 × 487 × 225 | 0.5 × 0.56 × | 50 | 40 | s | 5 |
| | mag) | | | 0.45 | | | | |

Notes for Table 2:
^{a} Samples were label-free fresh ex vivo tissue, unless otherwise noted.
^{b} For dual color acquisitions, the y-dimension is given as the final cropped y-dimension of one-color channel. The original y-dimension on the camera is >2x the final cropped y-dimension because color images are acquired simultaneously side-by-side along the y-axis on the camera. The x-dimension is given as the unskewed x-dimension of the acquired volume (# x-steps * x-step size).
^{c} Scans were acquired with a 70 mm focal length tube lens giving 4.66x effective magnification, unless otherwise noted.
^{d} Volume rate is reported if the scan type is a mirror-based roving scan. For mirror-based stationery and stage-scans the total acquisition type is given in seconds.
^{e} Laser power is typically for 488 nm laser excitation.

The MediSCAPE embodiments described herein were compared to confocal and two-photon microscopy. More specifically, MediSCAPE with 488 nm excitation; confocal with 488 nm and 561 nm excitation; and two-photon microscopy with 800 nm excitation were compared. To compare the autofluorescence contrast, fresh mouse colon mucosa and kidney samples were imaged with all three techniques. Cellular and tissue-level features were fairly similar across all three techniques, but point-scanning requires prohibitively long acquisition times for weak intrinsic fluorescence.

MediSCAPE's key advantages are its real-time 3D speeds, which facilitate in vivo and large area imaging, and its sensitivity, which allow the detection of weak intrinsic contrast. Here, we explain why MediSCAPE's speed and sensitivity are orders of magnitude better than that of point-scanning confocal and two-photon microscopy, which are the traditional techniques of choice for optically-sectioned fluorescence imaging. We also demonstrate that MediSCAPE's autofluorescence images of fresh tissue histoarchitecture are qualitatively similar to images acquired by confocal and two-photon microscopy at similar excitation and emission wavelengths.

Feasibility of Fast 3D Scanning for MediSCAPE vs Point-Scanning Confocal and Two-Photon

MediSCAPE's use of light sheet excitation leads to significant improvements in sensitivity thanks to parallelized excitation and emission detection of an entire plane in a tissue volume. This parallelization allows longer integration times and gentler laser excitation powers, which leads to reduced photobleaching and phototoxicity in tissues. On the other hand, confocal microendoscopy systems and bedside two-photon systems use point scanning, in which each individual pixel in the tissue volume is excited and captured sequentially. Point-scanning greatly decreases available integration time per pixel, while also requiring high galvanometer scanning speeds. Table 3 below shows the substantial difference between MediSCAPE's and a point-scanning microscope's galvanometer line scan rates and integration times per pixel for roughly equivalent volume imaging rates. Imaging parameters listed for MediSCAPE are from the first two datasets shown in Image Set 1, as an example.

TABLE 3
Comparison of imaging parameters on MediSCAPE and a
point-scanning microscope for equivalent volume rates

| | | | | | | | |
|---|---|---|---|---|---|---|---|
| Volume | | X | Y | Z | Galvo line | Pixel | Integration |
| Rate | FPS ³ | pixels | pixels | pixels | scan rate | rate | time per pixel |

Stationary, high-resolution scan

| | | | | | | | | | | | | |
|---|---|---|---|---|---|---|---|---|---|---|---|---|
| MediSCAPE¹ | 1.27 | Hz | 1021 | Hz | 802 | 615 | 250 | 1.27 | Hz | 157 MHz | 0.98 | ms |
| Point-scanning | 1.27 | Hz | 317 | Hz | 802 | 615 | 250 | 195 | kHz | 157 MHz | 6.3 | ns |

Roving, fast scan

| | | | | | | | | | | | | |
|---|---|---|---|---|---|---|---|---|---|---|---|---|
| MediSCAPE² | 9.3 | Hz | 1335 | Hz | 143 | 570 | 150 | 9.3 | Hz | 114 MHz | 0.75 | ms |
| Point-scanning | 9.3 | Hz | 1400 | Hz | 143 | 570 | 150 | 200 | kHz | 114 MHz | 8.8 | ns |

Notes for Table 3:
¹scan parameters based on mirror-based stationary scan of in vivo mouse kidney;
²scan parameters based on mirror-based roving scan of in vivo mouse kidney;
³ FPS is calculated for yz frames for MediSCAPE and xy frames for point-scanning.

For a single high-resolution scan in the mouse kidney, MediSCAPE took 0.79 sec to acquire a 802×615×250 xyz pixel volume of dual color autofluorescence contrast. To acquire an equivalent single-color volume at the same speed, a point-scanning microscope would need to scan its galvanometer at a line rate of 195 kHz, a speed that cannot be achieved even using resonant scanners. Moreover, the integration time per pixel would be 6.3 ns, which is close to many fluorophores' fluorescent lifetimes. In contrast, MediSCAPE's scanning mirror would need to move at only 1.27 Hz, while the integration time per pixel would be 0.98 ms. This 153,379 times longer integration time highlights why weak intrinsic contrast can be imaged with MediSCAPE so much more easily than by confocal and two-photon while maintaining reasonable laser power levels and acquisition times (see Hillman et al for additional supporting models). For a roving scan taken at 9.3 VPS, we see the same orders of magnitude difference between MediSCAPE and point scanning systems for the required galvanometer line scan rate and the resulting integration time per pixel.

Moreover, point-scanning microscopes designed for in vivo and bedside use require mirror scanning or physical movement of the probe or tissue to acquire z-stacks. This can be both mechanically challenging to implement and prone to motion artifact in the presence of in vivo tissue movement. MediSCAPE's lateral scanning and simultaneously recording from all depths at once removes this need to vary the microscope's focal depth during 3D imaging.

Comparison of Autofluorescence Contrast with MediSCAPE, Confocal and Two-Photon

To compare autofluorescence features captured by MediSCAPE to other microscopy techniques, sections of freshly excised mouse colon mucosa and kidney cortex were imaged with confocal, two-photon and MediSCAPE microscopes consecutively. Confocal imaging was performed on a Nikon inverted MR confocal. Two-photon imaging was performed using galvo-scanning and a Mai-Tai HP laser. Tissue was kept on ice between imaging sessions, kept moist with saline and imaged within three hours of excision.

Representative images from each tissue were captured and imaging parameters used to acquire each volume shown are given in Table 4. In fresh mouse colon mucosa, each imaging technique showed strong punctate green autofluorescence in the epithelial cells lining the crypts of Lieberkühn and diffuse red emission in the lamina propria in between crypt structures. Both MediSCAPE and confocal images were fairly similar, while two-photon excitation also revealed strong blue emission in the epithelial cells, as well as in fibers surrounding the crypts. In fresh mouse kidney cortex, all three imaging techniques were able to clearly visualize tubules through autofluorescence with proximal tubules showing higher emission at in the green channel and distal tubules showing higher emission in the red channel. Two-photon imaging also revealed blue autofluorescence in the tubules, overlapping highly with the green channel. Note that tubules here were imaged deeper into the kidney cortex and appear morphologically different from tubules closer to the cortex surface, as shown in MediSCAPE and confocal images. Also note that sample drift was a major issue in confocal and two-photon imaging over the course of volume acquisition in both tissues.

Although these datasets were acquired purely to compare contrast acquired on MediSCAPE to confocal and two-photon, imaging parameters shown in Table 4 show that confocal and two-photon imaging require almost two orders of magnitude more time to acquire a single-color volume of similar quality to images acquired using MediSCAPE in the same tissues.

TABLE 4
Imaging Parameters for SCAPE MediSCAPE, Confocal and Two-Photon

| | | |
|---|---|---|
| SCAPE | | |
| <0.45 | Confocal | 2P |
| (Nikon Plan Apo 10x + | 0.45 | 1.0 |
| Olympus XLUMPLFLN | (Nikon Plan Apo | (Olympus XLUMPLFLN |
| 20X) | 10x) | 20X) |

Colon Mucosa

| | | | |
|---|---|---|---|
| Size (x-y-z | 991 × 1000 × 263 | 1228 × 1228 × 102 | 510 × 510 × 160 |
| μm³) | | | |
| Size (x-y-z | 745 × 1000 × 240 | 1200 × 1200 × 37 | 600 × 600 × 80 |
| vox) | | | |
| # color | 2 | 2 | 3 |
| channels | | | |
| Total Time | 2 | 582 | 301 |
| (sec) | | | |
| Voxel Rate* | 179 MHz | 183 kHz | 287 kHz |
| Power at | 7.5 mW (488 nm) | 2.5 mW (488 nm) | 2.68 mW |
| sample | | 3.8 mW (561 nm) | (800 nm, 80 MHz) |

Kidney Cortex

| | | | |
|---|---|---|---|
| Size (x-y-z | 801 × 960 × 296 | 1228 × 1228 × 102 | 510 × 510 × 160 |
| μm³) | | | |
| Size (x-y-z | 801 × 691 × 250 | 1024 × 1024 × 37 | 600 × 600 × 80 |
| vox) | | | |
| # color | 2 | 2 | 3 |
| channels | | | |
| Total Time | 8 | 286 | 301 |
| (sec) | | | |
| Voxel Rate* | 34.6 MHz | 271 kHz | 287 kHz |
| Power at | <6 mW (488 nm) | 2.5 mW (488 nm) | 2.68 mW |
| sample | | 3.8 mW (561 nm) | (800, 80 MHz) |

*Voxel rate calculated as # of color voxels acquired per second of total imaging time

METHODS

In Vivo Mouse Tissue Preparation and Imaging

In vivo mouse imaging was carried out according to protocols reviewed and approved by Columbia University's Institutional Animal Care and Use Committee. Prior to imaging, a wild type mouse was heavily anesthetized using isoflurane and its snout was placed in a mouse-mask. Body temperature was maintained with a warming pad positioned on top of the mouse and breathing was monitored continuously. Abdominal organs were exposed first and the mouse was placed on a 60 mm diameter glass bottom dish mounted on a 3-axis stage. Organs were positioned to be against the surface of the glass coverslip for imaging from below. For roving imaging, the position of the mouse was translated during continuous imaging. Warm saline was used to periodically flush tissues to minimize drying and maintain body temperature. After organ imaging, the chest cavity was then opened and the heart was rapidly positioned for imaging prior to euthanasia.

For imaging brain microvasculature shown in Image Set 7 and Movie 10 (described above) , the mouse was anesthetized using urethane, and sealed bilateral glass cranial windows were implanted over the somatosensory cortices as previously described. A metal head plate was glued to the skull to enable head fixation under the MediSCAPE objective lens in an upright configuration. Imaging was performed following tail vein injection of ˜0.1 ml of 5% w/v 70,000 MW Fluorescein isothiocyanate˜dextran. The xyz position of the mouse was translated manually with 3-axis stages during roving imaging.

Fresh Mouse Tissue Preparation and Imaging

Fresh mouse tissue was excised from wild type mice according to protocols reviewed and approved by Columbia University's Institutional Animal Care and Use Committee. Mice were heavily anesthetized and then euthanized using cervical dislocation. Excised tissue was kept on ice until imaging or until at least 30 min before staining. All data was acquired within 3 hours of excision. Tissues were imaged from below in 30 mm diameter glass bottom dishes with the MediSCAPE objective in an inverted setup. Tissue was kept moist with saline and gently pressed down with a coverslip to create a flatter imaging surface when necessary. On the FIG. 2 system, tissues were placed in a petri dish and imaged from above (in an upright configuration). Tissues were kept moist with saline and pressed down with a coverslip when needed. For datasets showing stained fresh tissue, tissues were topically stained at room temperature for 1-3 minutes, rinsed with saline and imaged immediately, as described above.

Human Kidney Biopsy Collection and Imaging

De-identified fresh human kidney tissue was acquired through the Tissue Bank at the Columbia University Medical Center Department of Pathology under an IRB-approved protocol. Tissue was imaged within 24 hours of excision and stored in a petri dish with saline-soaked cloth at 4° C. and kept on ice before imaging. Tissue was imaged from below in a 30 mm diameter glass bottom dish with saline to keep moist.

Fresh Tissue Staining

Where indicated, tissues were topically stained with 0.01% proflavine (Sigma, 131105) in saline, 1% methylene blue (Ricca, 485016) and/or 0.01% fluorescein sodium in water. Dyes were gently applied with a cotton swab to tissue at room temperature for 1-3 minutes and then rinsed away 3× with saline. Stained regions were imaged immediately.

Histology

After imaging, all fresh tissues were marked with tissue marker to clearly indicate the face imaged on MediSCAPE, and placed into histology cassettes with the imaged face laid flat on biopsy paper. Tissues were fixed in 10% formalin for at least 24 hours at 4° C. Subsequent histological embedding, slicing, staining and mounting was done by Molecular Pathology Histology Services at the CUMC Herbert Irving Cancer Center. All mouse tissues were level cut into several 5μm flat face sections spanning the first 50-100μm of the imaged face and stained for H&E. Kidney biopsy tissue was cut into 2 μm flat face sections spanning the first 50-100 μm of the imaged face and stained for both H&E and PAS. Histology slides were digitally scanned using a Nikon AZ100 slide scanner. Regions of interest were matched by manually comparing structural features visible in MediSCAPE images and digital histology data.

Conclusion

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. An imaging apparatus comprising:
a first set of optical components having a proximal end and a distal end, wherein the first set of optical components includes a first objective disposed at the distal end of the first set of optical components, wherein the first objective has a magnification between 10× and 70× and a numerical aperture between 0.5 and 1.1;
a second set of optical components having a proximal end and a distal end, wherein the second set of optical components includes a second objective disposed at the proximal end of the second set of optical components;
a scanning element that is disposed proximally with respect to the proximal end of the first set of optical components and distally with respect to the distal end of the second set of optical components
wherein the scanning element is arranged to route excitation light through the first set of optical components in a proximal to distal direction so that the excitation light is projected into a sample that is positioned distally beyond the distal end of the first set of optical components,
wherein the excitation light that is projected into the sample forms a sheet of excitation light at an oblique angle, wherein a position of the sheet varies depending on an orientation of the scanning element,
wherein the first set of optical components routes detection light from the sample in a distal to proximal direction back to the scanning element, and
wherein the scanning element is further arranged to route the detection light so that the detection light will pass through the second set of optical components in a distal to proximal direction, so that the second set of optical components forms an intermediate image plane at a position that is proximally beyond the proximal end of the second set of optical components;
a folding mirror disposed proximally with respect to the proximal end of the first set of optical components and distally with respect to the distal end of the second set of optical components,
a light detector array; and
a third objective arranged to route light arriving from the intermediate image plane towards the light detector array.

2. The apparatus of claim 1, wherein the folding mirror is positioned between the scanning element and the distal end of the second set of optical components.

3. The apparatus of claim 1, wherein the first objective has a magnification between 50× and 70×, a numerical aperture between 0.9 and 1.1, and an effective focal length between 2.5 and 3.5 mm, and
wherein the second objective has a magnification between 40× and 60×, a numerical aperture between 0.65 and 0.85, and an effective focal length between 3 and 5 mm.

4. The apparatus of claim 3, wherein the first objective has a magnification of 60×, a numerical aperture of 1.0, and an effective focal length of 3 mm.

5. The apparatus of claim 3, wherein the second objective has a magnification of 50 ×, a numerical aperture of 0.75, and an effective focal length of 4 mm.

6. The apparatus of claim 3, wherein the first set of optical components includes at least one Plössl lens.

7. The apparatus of claim 3, wherein the first set of optical components comprises a 12.7 mm diameter 38.1 mm EFL achromat and a Plössl lens comprising two 12.7 mm diameter 50.8-mm-EFL achromats.

8. The apparatus of claim 3, wherein the second set of optical components includes at least one Plössl lens.

9. The apparatus of claim 3, wherein the second set of optical components comprises a Plössl lens made of two 1″ diameter 101.6-mm-EFL achromats and a 1″ diameter 76.2-mm-EFL achromat.

10. The apparatus of claim 3, wherein the first set of optical components comprises a telescope with a 1.5× magnification, and wherein the second set of optical components comprises a telescope with a 1.5× magnification.

11. The apparatus of claim 3, wherein the third objective has a magnification between 15× and 25× and a numerical aperture between 0.65 and 0.85.

12. The apparatus of claim 3, wherein the third objective has a magnification of 20× and a numerical aperture of 0.75.

13. An imaging apparatus comprising:
a first set of optical components having a proximal end and a distal end, wherein the first set of optical components includes a first objective disposed at the distal end of the first set of optical components;
a second set of optical components having a proximal end and a distal end, wherein the second set of optical components includes a second objective disposed at the proximal end of the second set of optical components;
a scanning element that is disposed proximally with respect to the proximal end of the first set of optical components and distally with respect to the distal end of the second set of optical components
wherein the scanning element is arranged to route excitation light through the first set of optical components in a proximal to distal direction so that the excitation light is projected into a sample that is positioned distally beyond the distal end of the first set of optical components,
wherein the excitation light that is projected into the sample forms a sheet of excitation light at an oblique angle, wherein a position of the sheet varies depending on an orientation of the scanning element,
wherein the first set of optical components routes detection light from the sample in a distal to proximal direction back to the scanning element, and
wherein the scanning element is further arranged to route the detection light so that the detection light will pass through the second set of optical components in a distal to proximal direction, so that the second set of optical components forms an intermediate image plane at a position that is proximally beyond the proximal end of the second set of optical components;
a light detector array;
a third objective arranged to route light arriving from the intermediate image plane towards the light detector array; and
an optically transparent spacer positioned and configured to cover the first objective and to press against tissue being imaged.

14. The apparatus of claim 13, wherein the optically transparent spacer sets a working distance for the first objective to capture a 50-350 μm depth range into the tissue.

15. The apparatus of claim 13, wherein the optically transparent spacer is incorporated into a cap that provides a watertight seal between the optically transparent spacer and a distal end of the first objective.

16. The apparatus of claim 15, further comprising a quantity of a medium positioned between the optically transparent spacer and the first objective, wherein the medium has a refractive index selected to match an immersion medium of the first objective, and wherein the quantity of medium optically couples the optically transparent spacer to the first objective, and wherein the cap provides a water-tight seal.

17. The apparatus of claim 13, wherein the optically transparent spacer is formed from a solid medium with a refractive index matching a required immersion medium of the first objective.

18. The apparatus of claim 13, wherein the optically transparent spacer has an external surface positioned between 25 and 250 μm proximal to a primary focal plane of the first objective.

19. The apparatus of claim 13, wherein the optically transparent spacer permits fast 3D imaging of a sample that is gradually moved across an external surface of the spacer permitting stitching of a contiguous 3D image of the sample.

20. The apparatus of claim 13, wherein the wherein the first objective has a magnification between 10× and 70× and a numerical aperture between 0.5 and 1.1.
